(19) United States
(12) Patent Application Publication
(10) Pub. No.: US 2023/0347057 A1
(43) Pub. Date: Nov. 2, 2023

(54) VACUUM GENERATION DEVICES AND METHODS

(71) Applicant: YourBio Health, Inc., Medford, MA (US)

(72) Inventors: Vincent J. Barone, Medford, MA (US); Li Yang Chu, Medford, MA (US); Eric Nicholas Ambos, Medford, MA (US); Paul F. Bente, IV, Medford, MA (US)

(73) Assignee: YourBio Health, Inc., Medford, MA (US)

(21) Appl. No.: 17/920,830

(22) PCT Filed: Apr. 30, 2021
(86) PCT No.: PCT/US2021/030262

§ 371 Date: Oct. 24, 2022

Related U.S. Application Data
(60) Provisional application No. 63/019,243, filed on May 1, 2020.

(51) Int. Cl. A61M 5/20; A61M 5/315

(52) U.S. Cl. CPC A61M 5/2053; A61M 5/31511

(57) ABSTRACT

The present disclosure generally relates to vacuum generation devices.

### CROSS-REFERENCE TO RELATED APPLICATIONS

This application claims the benefit of U.S. Provisional Patent Application Ser. No. 63/019,243, filed May 1, 2020. This application is incorporated herein by reference.

### FIELD

The present disclosure generally relates to devices and methods for generating vacuum.

### SUMMARY

In one aspect of the disclosure, a vacuum generation device is provided. The device may include a chamber having an active volume that is decreasable from a first volume to a second volume due to application of force during a priming step. The priming step may have a first phase and a second phase, and the chamber may be biased to return toward the first volume when the application of force ceases. A required application of force may be higher in the first phase than in the second phase. The device may include a one-way vent, where air exits the chamber through the one-way vent as the active volume is decreased from the first volume to the second volume. Return of the chamber toward the first volume from the second volume may create a vacuum. In some embodiments, the chamber does not include a flexible dome, e.g., one that is biased to return toward a non-compressed state.

In another aspect of the disclosure, a method of generating vacuum is provided. The method may include applying a first force to decrease an active volume of a chamber from a first volume during a first phase of a priming step and applying a second force to further decrease the active volume of the chamber during a second phase of the priming step. The first force may be greater than the second force. The method may include venting air out of the chamber during the priming step. The method may include ceasing application of force to permit the active volume of the chamber to return toward the first volume, wherein vacuum is generated due to the return of the active volume of the chamber toward the first volume.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments that incorporate one or more aspects of the disclosure will be described by way of example with reference to the accompanying figures, which are schematic and are not necessarily intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic of a vacuum generation device in accordance with aspects of the disclosure.

FIG. 2 is a schematic of another embodiment of a vacuum generation device.

FIG. 3 is a schematic of another embodiment of a vacuum generation device.

FIG. 4 is a schematic of another embodiment of a vacuum generation device.

FIG. 5 is a cross section of another embodiment of a vacuum generation device, the device being in a ready stage.

FIG. 6 is a cross section of the vacuum generation device of FIG. 5 in stage 1: a first phase of a priming step.

FIG. 7 is a cross section of the vacuum generation device of FIG. 5 between stage 1 and stage 2 in a second phase of the priming step.

FIG. 8 is a cross section of the vacuum generation device of FIG. 5 in stage 2: the end of the priming step.

FIG. 9 is a cross section of the vacuum generation device of FIG. 5 in stage 3: a first part of a vacuum generation step.

FIG. 10A is an enlarged portion of detail A of the vacuum generation device of FIG. 9.

FIG. 10B is an enlarged portion of detail B of the vacuum generation device of FIG. 9.

FIG. 11 is a cross section of the vacuum generation device of FIG. 5 in stage 4: the end of the vacuum generation step.

FIG. 12 depicts a graph of force and pressure profiles of a vacuum generation device through each operational stage.

FIG. 13 is a graph of force and pressure profiles of another vacuum generation device through each operational stage.

FIG. 14 is a cross section of another vacuum generation device, the device being in a ready stage.

FIG. 15 is a cross section of the vacuum generation device of FIG. 14 in stage 1: a first phase of a priming step.

FIG. 16 is a cross section of the vacuum generation device of FIG. 14 in stage 2: a second phase of the priming step.

FIG. 17 is a cross section of the vacuum generation device of FIG. 14 in stage 3: a first phase of a vacuum generation step.

FIG. 18 is a cross section of the vacuum generation device of FIG. 14 in stage 4: the end of the vacuum generation step.

### DETAILED DESCRIPTION

Aspects of the disclosure are not limited in application to the details of construction and the arrangement of components set forth in the following description or illustrated in the drawings. Also, the phraseology and terminology used herein is for the purpose of description and should not be regarded as limiting.

In some embodiments, a vacuum generation device includes an actuation mechanism that is designed such that the actuation mechanism is harder to manipulate during a first phase of actuation, and easier to manipulate during a final phase of actuation.

In some embodiments, a locking arrangement may be employed to achieve such an actuation profile.

In some embodiments, a vacuum generation device may include an actuation mechanism that is designed such that a first return element is loaded during a first phase of a priming step, and a second return element is loaded during a second phase of a priming step, where a stiffness of the first return element is higher than a stiffness of the second return element. A locking arrangement may be employed to separate the first and second return elements such that they are loaded independently of one another and/or unloaded independently of one another, as opposed to being serially loaded/unloaded.

In some embodiments, the vacuum generation device includes a chamber having an active volume that can be controllably changed. The vacuum generation device may produce a negative pressure differential relative to ambient pressure (e.g., a vacuum) by increasing the active volume of the chamber while keeping the active volume sealed relative to ambient pressure. In one set of embodiments, the chamber does not include a flexible dome that is biased to return toward a non-compressed state. In another set of embodiments, however, the chamber does include such a dome.

In some embodiments, the vacuum generation device may be configured such that, prior to actuation of the vacuum generation device, the active volume of the chamber is at an initial volume. Actuation of the vacuum generation device may cause the active volume of the chamber to decrease to a second volume that is smaller than the initial volume. In some embodiments, air inside the chamber is expelled as the active volume is decreased, e.g. through a one-way valve. This action of decreasing the active volume of the chamber may be referred to as priming.

Different user actions or gestures could be used to achieve the priming step. Examples include: pressing, squeezing, twisting, pulling, and/or pinching. For the priming step, a user may apply a force directly to the chamber itself, or to an actuator that transmits force to the chamber. In some embodiments, the force applied by the user to the actuator is mechanically transmitted to the chamber. In some embodiments, the vacuum generation device may generate vacuum by means of human-applied force.

In some embodiments, the vacuum generation device is purely mechanical. In other embodiments, however, the vacuum generation device may include electrical components, such as a controller, or a motor, etc.

After priming has completed, a return force may be applied to the chamber to cause the active volume of the chamber to return back towards (but not necessarily fully return in all embodiments) the initial, larger volume. As a result, as discussed above, this increase in the active volume of the chamber may generate a vacuum. This action of increasing the active volume of the chamber may be referred to as a return.

In some embodiments, the chamber is configured to be biased to return toward a configuration that increases the active volume of the chamber. This biasing force may serve as the return force for expansion. In some embodiments, a portion of the chamber may be coupled to, or made of, a potential energy storing material or element, such as a spring. For example, at least a portion of a chamber wall may be formed from an elastic material, or a moveable portion of the chamber wall may be coupled to an elastic mechanism. In some embodiments, when the applied force to the device from the user during priming ceases or is decreased, the chamber may automatically begin to revert toward its original volume, which may result in vacuum generation.

It is recognized that, in some embodiments, the vacuum generated by the return of the chamber towards its original volume may resist the return of the chamber towards its original volume. This may limit the amount of vacuum that can be generated by the chamber.

Accordingly, the potential need is recognized, in some embodiments, for a feature that helps to promote return of the chamber back towards its original volume.

In some embodiments, the chamber is designed such that the active volume is harder to decrease (e.g. requires more applied force from a user) during a first phase of priming, and easier to decrease (e.g. requires less applied force from a user) during a later or final phase of priming. This may result in a return force that is lower during a first phase of return (when the force resisting return is the lowest) and a return force that is higher during a later or final phase of return (when the force resisting return is highest).

The chamber may comprise any suitable arrangement, such as a syringe, a container with a piston or other moveable wall, where the position of the moveable wall determines the active volume of the chamber.

In one set of embodiments, the chamber does not include a flexible dome that is biased to return toward a non-compressed state. In another set of embodiments, however, the chamber does include such a dome.

In some embodiments, the biasing force is provided by more than one spring or other potential energy storing material or element. In some embodiments, the biasing force is provided by a first spring and a second spring arranged in series. The first and second springs may have different stiffnesses.

In some embodiments, the first and second springs may be separated by a locking arrangement. During a first phase of the priming step, the locking arrangement may permit compression of the first spring and prohibit compression of the second spring. During a second phase of the priming step, the locking arrangement may prohibit compression of the first spring and permit compression of the second spring. In some embodiments, the spring constant of the first spring may be greater than the spring constant of the second spring.

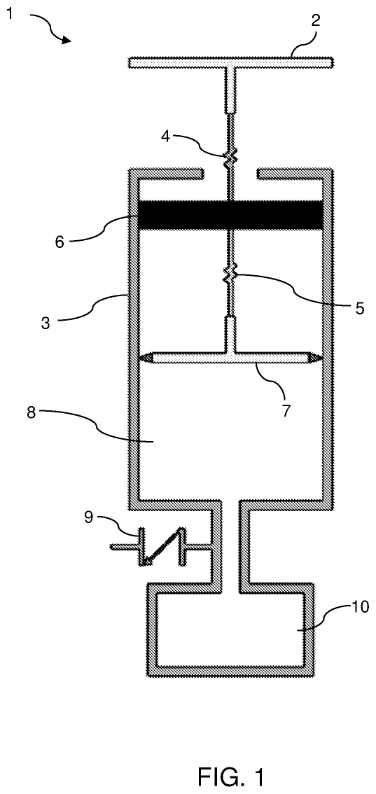

One illustrative embodiment of a vacuum generation device 1 is shown in FIG. 1 as a non-limiting example. The device 1 in this figure comprises a syringe with an actuator 2, a syringe body 3, and a piston 7 that is moveable through the syringe body 3 to define an active volume 8. The actuator 2 may be coupled to a first spring 4 and a second spring 5. The first spring 4 may have a first spring constant, and the second spring 5 may have a second spring constant, where the first and second spring constants may be different. In some embodiments, the first spring constant may be greater than the second spring constant. The springs 4, 5 may be arranged in series within the syringe body 3 and are separated by a locking arrangement 6. The locking arrangement 6 may be mechanical and/or electrical.

To generate vacuum, a user may apply a force to the actuator 2 (e.g. by pressing down on the actuator 2). The user's applied force may be transmitted through the first and second springs to move the piston 7.

When actuated, the locking arrangement 6 between the two springs 4, 5 may initially allow compression of the first spring 4. After the first spring 4 has been compressed a certain distance, the locking arrangement 6 may prevents further compression of the first spring 4, and may only permit compression of the second spring 5.

As the piston 7 moves downwardly to decrease the active volume 8 during this priming phase, air within the active volume may exit the active volume via a one-way valve 9.

At the completion of the priming phase, the actuator 2 may be allowed to return toward the original starting position, e.g. due to cessation of user-applied force to the actuator 2. This movement, in conjunction with the closing of the one-way valve 9, may generate a vacuum within the active volume, and the vacuum may be experienced at a downstream location that is in fluid communication with the active volume 8, such as buffer volume 10. In some embodiments, the device is configured to abut against and seal against skin to apply vacuum to the skin. The buffer volume may serve to represent a combination of the skin-interfacing portion of the device with the skin forming an airtight chamber to which vacuum can be applied.

During the return, the first spring 4 may be prohibited by the locking arrangement 6 from decompressing, while the second spring 5 may be permitted to decompress a set distance, which may be less than or equal to the distance it was compressed. When the second spring 5 has decompressed to the point where the force exerted on the piston 7 due to the second spring 5 is equal to the force exerted on the piston 7 due to the vacuum that is generated, the locking arrangement 6 may prevent further compression or decompression of the second spring 5 and only permit decompression of the first spring 4. The first spring may decompress to the point where the force exerted on the piston 7 by the first spring 4 is equal to the force exerted on the piston 7 by the vacuum that is generated. At this point, actuation of the device 1 and target vacuum generation is complete.

In some embodiments, the actuator 2 does not reach its actual original starting position, but may come close to the original starting position. In other embodiments, the actuator 2 does return to its original starting position.

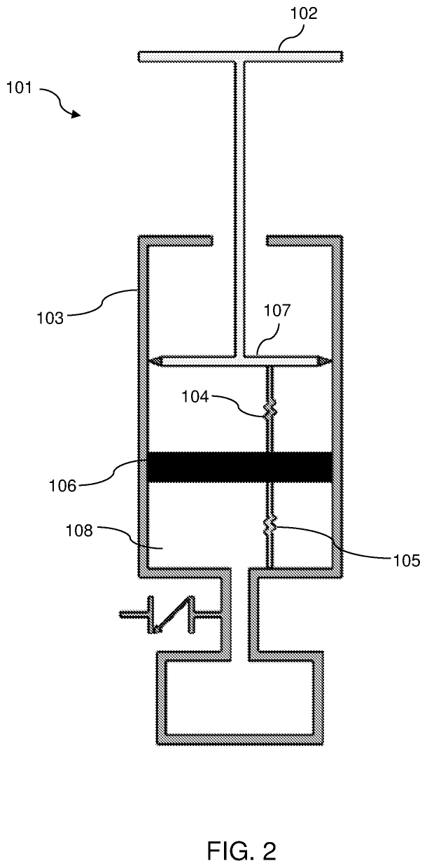

Another embodiment of a vacuum generation device is shown in FIG. 2. The device 101 in this figure comprises an actuator 102, a housing 103, and a piston 107 that is moveable through the housing 103 to define an active volume 108. The actuator 102 may be coupled to a first spring 104 and a second spring 105. The first spring 104 may have a first spring constant, and the second spring 105 may have a second spring constant, where the first and second spring constants may be different. In some embodiments, the first spring constant may be greater than the second spring constant. The springs 104, 105 may be arranged in series within the housing 103 and are separated by a locking arrangement 106. The locking arrangement 106 may be mechanical and/or electrical. The first spring 104 is positioned between the piston 107 and the locking arrangement 106, while the second spring is positioned between the locking arrangement 106 and the housing 103. In this illustrative embodiment, the first and second springs 104, 105 are loaded via compression as the device is actuated and the piston 107 is moved downward.

The locking arrangement 106 may serve to separate the loading of the first and second springs. During a first phase of a priming step, the locking arrangement may permit compression of the first spring and prohibit compression of the second spring. During a second phase of the priming step, the locking arrangement may prohibit compression of the first spring and permit compression of the second spring.

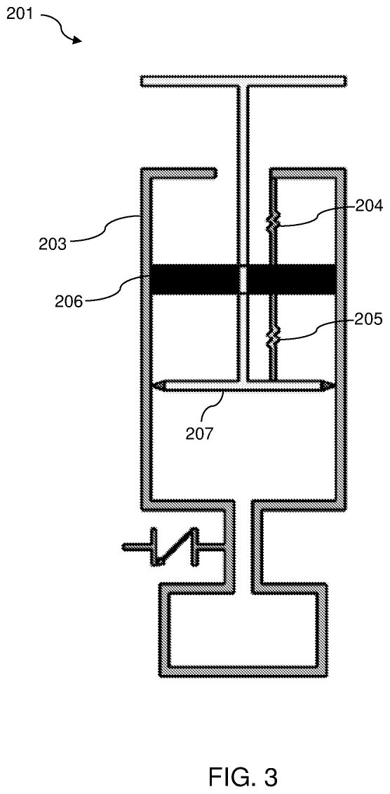

It should be appreciated that springs may be loaded in ways other than compression, such as extension. As an example, another embodiment of a vacuum generation device that loads springs in extension is shown in FIG. 3.

The device 201 in FIG. 3 includes a first spring 204 and a second spring 205 separated by a locking arrangement 206. The first spring 204 is positioned between the housing 203, while the second spring 205 is positioned between the locking arrangement 206 and the piston 207. In this illustrative embodiment, the first and second springs 204, 205 are loaded via extension as the device is actuated and the piston 207 is moved downward.

The locking arrangement 206 may serve to separate the loading of the first and second springs. During a first phase of a priming step, the locking arrangement may permit elongation of the first spring and prohibit elongation of the second spring. During a second phase of the priming step, the locking arrangement may prohibit elongation of the first spring and permit elongation of the second spring. In some embodiments, the spring constant of the first spring may be greater than the spring constant of the second spring.

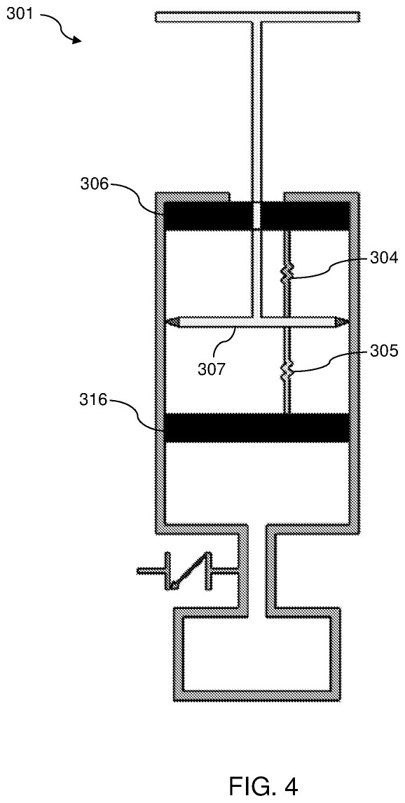

In some embodiments, a combination of loading arrangements may be used, e.g. one or more springs may be loaded in compression, while one or more other springs may be loaded in extension. As an example, another embodiment of a vacuum generation device that loads one spring in compression and one spring in extension is shown in FIG. 4.

The device 301 in FIG. 4 includes a first spring 304 and a second spring 305. The first spring 304 is positioned between a first locking arrangement 306 and the piston 307, while the second spring 305 is positioned between the piston 307 and a second locking arrangement 316. In this illustrative embodiment, the first spring 304 is loaded via extension as the device is actuated and the piston 307 is moved downward, while the second spring 305 is loaded via compression.

The locking arrangements 306, 316 may serve to separate the loading of the first and second springs. During a first phase of a priming step, the first locking arrangement 306 may permit elongation of the first spring while the second locking arrangement 316 may prohibit compression of the second spring 305. During a second phase of the priming step, the first locking arrangement may 306 prohibit elongation of the first spring 304 and the second locking arrangement 316 may permit elongation of the second spring 305. In some embodiments, the spring constant of the first spring may be greater than the spring constant of the second spring.

The locking arrangements in various embodiments may interact with the housing, the actuator, and/or the return elements in order to separate the loading of the return elements from one another during the priming step, and to separate the unloading of the return elements from one another during the vacuum generation step.

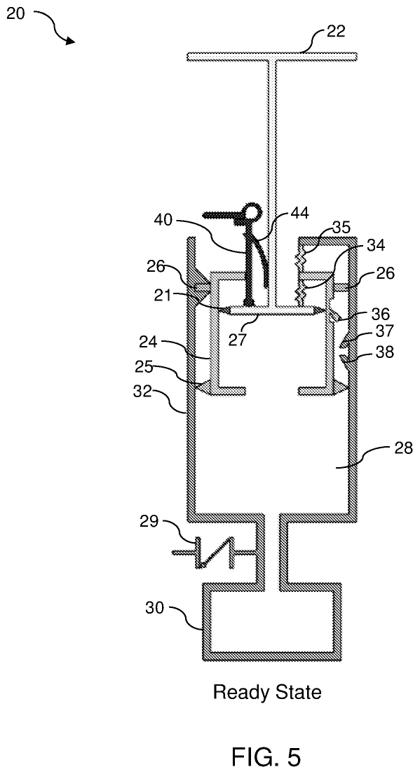

Another embodiment of a vacuum generation device is shown in FIG. 5. A user may manually prime the device by pushing on an actuator 22. According to one aspect, the priming step may have a first phase and a second phase. In some embodiments, the force required to actuate the actuator is higher in the first phase than in the second phase. Alternatively or in addition, in some embodiments, a first return element is loaded in the first phase and a second return element is loaded in the second phase, wherein a stiffness of the first return element is higher than a stiffness of the second return element.

In the embodiment of FIG. 5, during priming, springs 34 and 35 are loaded. The vacuum generation device 20 includes a locking arrangement that separates the loading of the first spring 34 from the loading of the second spring 35. The second spring 35 has a lower stiffness than the first spring 34. The locking arrangement may be configured to permit the stiffer spring to be loaded first during the first phase of a priming step, while prohibiting the less stiff spring from being loaded. During the second phase of the priming step, the locking arrangement may permit the less stiff spring to be loaded. In some embodiments, during the second phase of the priming step, the stiffer spring discontinues further loading while the less stiff spring is loaded. As a result, during the priming step, the force required to actuate the actuator may be higher in the first phase than in the second phase.

In the embodiment of FIG. 5, the locking arrangement that separates the loading of the springs includes friction pads 26 and swing latch 40. The friction pads 26 are positioned between an outer housing 32 and an inner housing 24. The friction pads may be fixed to the outer housing 32 while the inner housing 24 may be configured to slide against the friction pads 26 during the second phase of the priming step. Alternatively, the components may be reversed such that the friction pads are fixed to the inner housing 24 while the friction pads 26 may be configured to slide against the outer housing during the second phase of the priming step. The swing latch 40 may be fixed to a piston 27 and may engage with the inner housing 24 at the end of the first phase of the priming step. The function of the friction pads 26 and the swing latch 40 is discussed in more detail below.

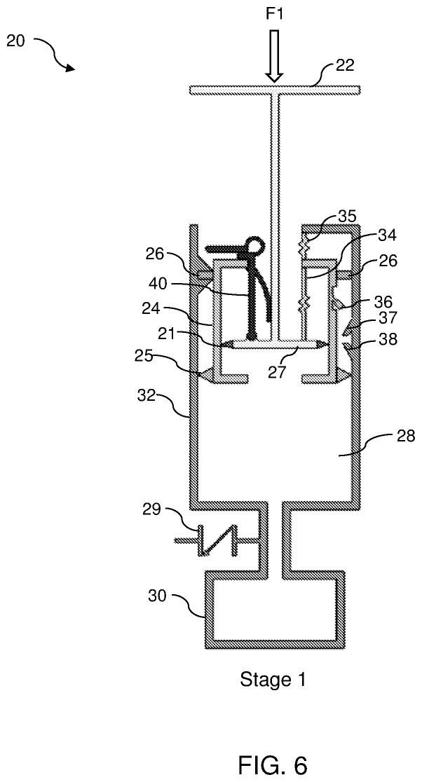

FIGS. 5-11 depict the vacuum generation device 20 undergoing a series of stages as the device is actuated and generates vacuum. In FIG. 5, the device 20 is shown in a ready state. A user manually exerts an actuation force F1 upon actuator 22, e.g. by pushing down with one or more fingers or a palm. As shown in FIG. 6, which depicts stage 1, this actuation force F1 causes the piston 27 to move relative to the inner housing 24 in a direction that decreases the active volume 28 (downward in FIG. 6). The piston may include a seal 21 that seals the piston against the housing 32. Pressure buildup from movement of the piston 27 is relieved via a one-way valve 29. The inner housing 24 is held in place by the friction pads 26, and does not move during this first phase of the priming step. The first spring 34 is fixed to the piston 27 and to the inner housing 24. Movement of the piston 27 relative to the inner housing 24 results in elongation of the first spring 34, thus loading the first spring 34. The retaining force exerted by the friction pads against the inner housing 24 is greater than the force required to elongate the spring 34, and thus the first spring 34 elongates while the friction pads 26 prevent movement of the inner housing.

As the piston 27 moves downwards, the swing latch 40, which is fixed to the piston 27, also moves downwards until the swing latch 40 engages with the inner housing 24, as shown in FIG. 6 (stage 1). Engagement of the swing latch 40 with the inner housing constrains further relative movement between the piston 27 and the inner housing 24, thus locking the first spring 34 in a loaded state, and also preventing further loading of the spring 34. With the piston 27 locked to the inner housing 24 due to engagement of the swing latch 40 with the inner housing 24, force F1 on the actuator 22 overcomes the retaining force of the friction pads 26, thus freeing the inner housing 24 to move relative to the outer housing 32. As a result, the first spring 34 is locked, and the second spring 35, which is fixed between the inner housing 24 and the outer housing 32, may begin to load. Locking of the first spring 34 completes the first phase of the priming step, and enablement of loading of the second spring 35 begins the second phase of the priming step.

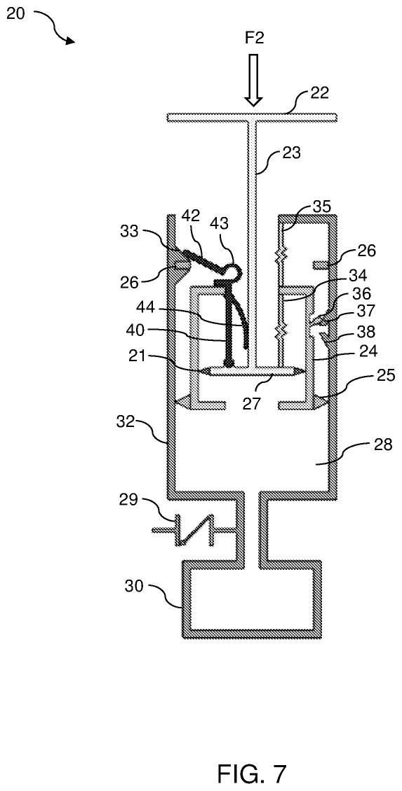

FIG. 7 depicts the vacuum generation device between stage 1 and stage 2, in a second phase of the priming step. The user manually exerts an actuation force F2 upon actuator 22. In this second phase of the priming step, with the piston 27 locked to the inner housing 24 due to engagement of the swing latch 40 to the inner housing 24, displacing the piston 27 causes the inner housing 24 to move with the piston 27, which in turn causes the second spring 35 to elongate, thus loading the second spring 35. In some embodiments, the amount of force F2 required to further displace the piston 27 in this phase of the priming step may be less than the force F1 required to displace the piston 27 in the first phase of the priming step depicted in FIG. 6. This may be due, at least in part, to the second spring 35, which may have a lower stiffness than that of the first spring 34. The inner housing 24 may include a seal 25 that permits the inner housing 24 to seal against the outer housing 32.

However, it should be appreciated that, in other embodiments, the force F2 may be equal to or greater than the force F1.

As also seen in FIG. 7, a latch arm 42 of swing latch 40, which has been displaced downwards with the piston 27, abuts against a latch ramp 33 on the outer housing 32. The swing latch 40 also includes a spring arm 44 that engages with the piston shaft 23 and prevents the swing latch 40 from unlatching. A hinge 43 of the swing latch 40 permits the latch arm 42 to flex upwards, allowing the latch arm 42 to slide past the latch ramp 33.

In addition, as seen in FIG. 7, a ratchet lock 36 on the inner housing 24 engages an upper ratchet ramp 37 on the outer housing 32. As seen in FIG. 6, the ratchet lock 36 is biased downward. Engagement of the ratchet lock 36 with the upper ratchet ramp 37 moves the rachet lock from a biased downward angle to an upward angle, allowing movement past the lower ratchet ramp 38.

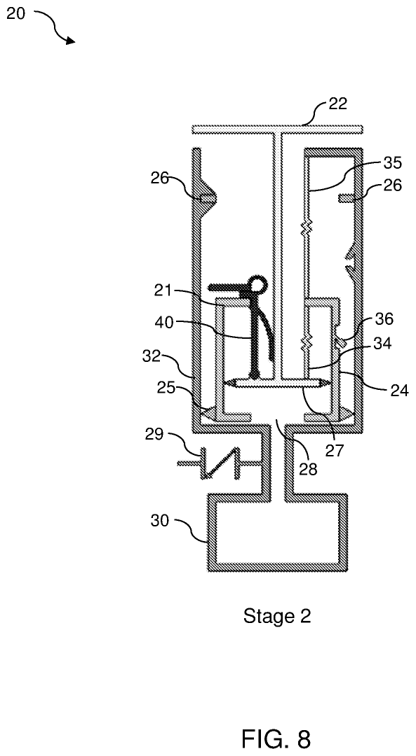

The continued application of actuation force F2 moves the device to the end of the priming step, which is stage 2, depicted in FIG. 8. In this state, the second spring 35 has been further loaded. In some embodiments, the second spring 35 has reached its full extension length. The swing latch 40 has returned to its original closed state, and the ratchet lock 36 returns to its biased downward state. In the illustrative embodiment of FIG. 8, the inner housing 24 has bottomed out against the outer housing.

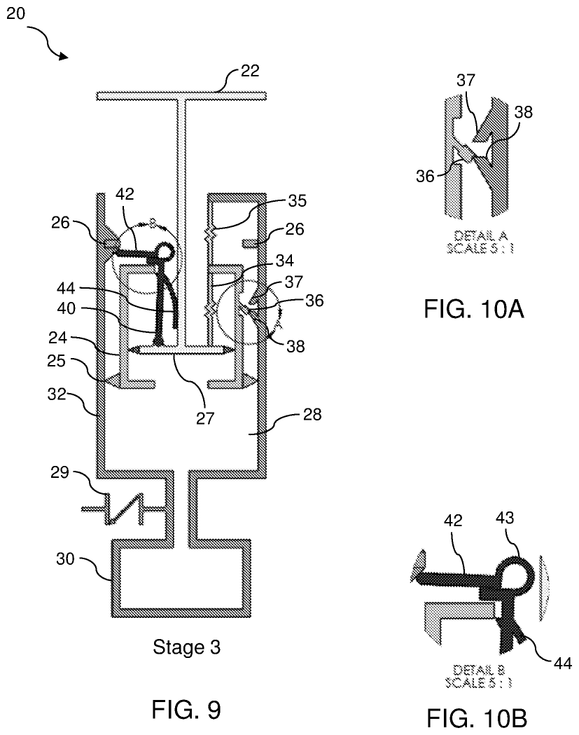

Next, the user ceases applying force to the actuator, transitioning the device to the vacuum generation step, which is stage 3, depicted in FIG. 9. With the removal of application of force on the actuator, the second spring 35 retracts, causing the piston 27 and the inner housing 24 begin to move back in the direction that increases the active volume 28 (upward in FIG. 9). Vacuum in the active volume 28 is generated as the volume of active volume 28 increases, and the one-way valve 29 does not permit entry of air into the active volume 28. The buffer volume 30, which is in fluid communication with the active volume 28, is thus subjected to vacuum.

As the inner housing 24 retracts upward, the ratchet lock 36 engages the lower ratchet ramp 38, preventing re-extension of the second spring 35, as shown in the detailed view of FIG. 10A. The swing latch 40 abuts against the latch ramp, causing the swing latch spring arm 44 to be overcome. As a result, the swing latch 40 disengages from the inner housing 24, as shown in the detailed view of FIG. 10B. With the swing latch 40 disengaged from the inner housing 24, the piston 27 is permitted to move relative to the inner housing 24, thus permitting the first spring 34 to retract. Retraction of the first spring 34 retracts the piston 27.

In some embodiments, the device is configured such that the ratchet lock 36 is positioned to engage the lower ratchet ramp 38 when the spring force from the second spring 35 approaches the force of the generated vacuum.

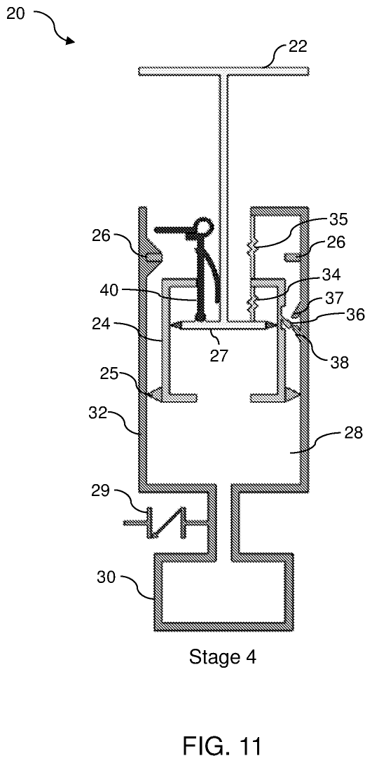

FIG. 11 depicts the device in its final stage, stage 4: the first spring 34 has completed decompression and the device has achieved a target vacuum level in the buffer volume 30. The vacuum level may be at its greatest as the device approaches the end of stage 4, and in turn may give rise to the highest force acting against the return of the piston toward its original position.

In some embodiments, using a higher stiffness spring or return element to retract the piston at the end of the vacuum generation step as compared to the start of the vacuum generation step may help to counteract the higher vacuum force, and may help to return the piston to a position closer to the original piston position, which in turn may help generate yet more vacuum.

In some embodiments, using a spring/return element with a lower stiffness at the start of the vacuum generation step and a spring/return element with a higher stiffness at the end of the vacuum generation step may help to counteract the higher vacuum force occurring at the end of the vacuum generation step, and may help to return the piston to a position closer to the original piston position, which in turn may help generate yet more vacuum.

In some embodiments, using a spring/return element with a lower peak reaction force at the start of the vacuum generation step and a spring/return element with a higher peak reaction force at the end of the vacuum generation step may help to counteract the higher vacuum force occurring at the end of the vacuum generation step, and may help to return the piston to a position closer to the original piston position, which in turn may help generate yet more vacuum.

It should be appreciated that many different components can serve as the first and second springs, such as: coil springs, serpentine springs, cantilevered springs, leaf springs, volute springs, pneumatic springs, elastomer springs, torsion springs, stretchable elastomers (e.g. rubber bands), compressible foam. In some embodiments, one or both of the springs may be a torsional spring. A vacuum generation device utilizing a torsional spring may also include a rotational arrangement that loads and unloads the torsional spring.

It should be appreciated that different types of components can serve as return elements that move a piston back towards a starting position in order to generate vacuum. One or both of the springs may be substituted with other types of components that may serve as return elements, such as servos, rotary or linear electric motors, electromechanical devices, or other suitable devices. In some embodiments, one or both of the springs may be substituted with a return element comprising a bi-stable element, e.g. a bi-stable dome such as a snap dome. The bi-stable element may be configured to move from a first stable state to a second stable state in response to an applied force that exceeds a trip force of the bi-stable element. The bi-stable element may be incapable of moving from the second stable state back to the first stable state in the absence of an external force on the bi-stable element. In other words, the bi-stable element may not be biased to return to the first stable state, and instead may remain in the second stable state until an external force is applied to the bi-stable element to move the bi-stable element back to the first stable state.

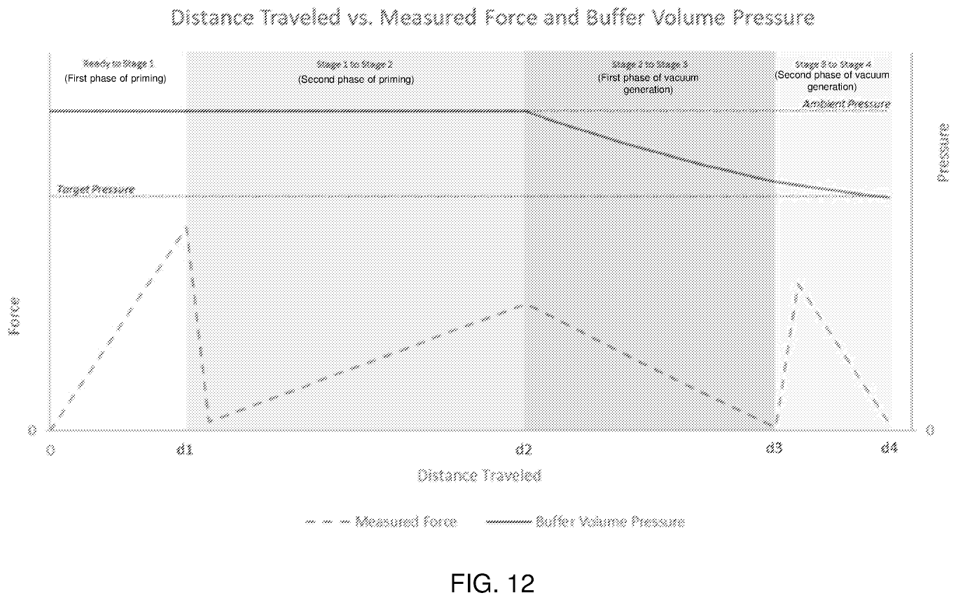

FIG. 12 depicts a graph of force and pressure profiles of a vacuum generation device through each operational stage.

The graph may, for example, represent the force and pressure profiles of the vacuum generation device 20 through each operational stage.

Distance traveled is plotted on the X-axis. Distance traveled reflects a magnitude of the distance traveled by a piston or other airtight, volume-separation component. In moving from distance 0, to d1, to d2, the piston moves in a direction that decreases the active volume. In moving from d2 to d3 to d4, the piston reverses and moves in a direction that increases the active volume. As seen in FIG. 12, in the vacuum generation step (stage 2 to stage 4), the piston reverses movement back toward its original starting position. However, the distance traveled in this step continues to increase. As such, distance traveled on the graph is in contrast to position. A plot of position would instead show the position decreasing in the vacuum generation step.

The dash-dash curve represents measured force, and is a plot of force (left-side Y-axis) versus distance traveled (X-axis). Measured force is what would be measured by a force probe on top of the actuator, where the probe is used to actuate the device. In the priming step, the measured force is the reaction force of the springs or other return elements as they are loaded. In the vacuum generation step, the measured force is the reaction force of the springs or other return elements less the reaction force of the generated vacuum. During the vacuum generation phase, the generated vacuum produces a reaction force that tends to resist return of the piston or other airtight, volume-separation component back towards its pre-actuated position. Thus, the reaction force of the generated vacuum may work against the springs or other return elements that bias the piston or other airtight, volume-separation component back towards its pre-actuated position.

The solid curve represents pressure in the buffer volume, and is a plot of pressure (right-side Y-axis) versus distance traveled (X-axis). For example, in the vacuum generation device 20, the pressure experienced in the buffer volume 30. Two reference pressures are noted on the graph: ambient pressure and a target pressure. A buffer volume pressure that crosses below the ambient pressure reference line signifies that the buffer volume is under vacuum. The line moving further below the ambient pressure reference line represents greater vacuum being generated. The target pressure represents a certain level of vacuum that is desired to be achieved by the vacuum generation device. A buffer volume pressure that crosses below the target pressure reference line signifies that the vacuum generation device has generated the desired vacuum level.

In the first phase of priming, in which the device moves from the ready state to stage 1, the measured force increases as the first spring (or other return element) is loaded. The slope of the measured force in the first phase of priming may reflect the stiffness of the first spring. In the transition to the second phase of priming, the measured force drops suddenly. This may reflect the device transitioning from loading the first spring (or other return element) to loading a second spring (or other return element). As discussed above, a locking arrangement may be used to separate the first spring from the second spring such that they are independently loaded (as opposed to being loaded serially). In some embodiments, the locking arrangement may lock the second spring from being loaded as the first spring is loaded in the first phase of priming. Then, during the second phase of priming, the locking arrangement may lock the first spring from being loaded and permit the second spring to be loaded. As shown in FIG. 12, the drop in measured force between the first and second phases of priming represents the first spring being locked out, and thus the reaction force from the first spring is no longer experienced by the force probe on the actuator (and, similarly, a user pushing on the actuator may also experience a drop in resistance).

After this drop, the measured force may proceed to steadily climb in the second phase of priming, which may represent the loading of the second spring. The slope of the measured force in the second phase of priming may reflect the stiffness of the second spring. As seen in FIG. 12, the slope of the measured force curve in the first phase of priming may be steeper than the slope of the curve in the second phase of priming, which may indicate that the stiffness of the first spring is greater than the stiffness of the second spring.

As also seen in FIG. 12, the peak force during the second phase of priming is lower than the peak force during the first phase of priming. As a result, a user actuating a force on the actuator may feel that the first phase of priming requires a greater exertion of force than the second phase of priming.

Moving next to the vacuum generation step of the measured force curve, the measured force curve has a second peak and begins to drop as the device moves from stage 2 to stage 3. The measured force decreases as the device moves through the first phase of vacuum generation due to unloading of the second spring. The return force of the second spring is greatest initially, and decreases as the spring unloads. As the second spring unloads, a vacuum is generated. This is evidenced by the buffer volume pressure curve, which crosses to be under ambient pressure as the device transitions from stage 2 to stage 3. The reaction force of the vacuum acts against the return force of the spring, thus pulling the magnitude of the measured force curve downward. In other words, without any vacuum generation, one would expect the force profile to mirror the loading of the spring in the second phase of priming. Instead, the measured force drops more quickly due to the generation of vacuum. As shown in FIG. 12, just as the net return force is about to approach zero, the measured force rebounds again to a third peak. This is due to the device transitioning between the first phase of vacuum generation to the second phase of vacuum generation, in which the second spring is locked by the locking arrangement, and the first spring is unlocked by the locking arrangement, thus permitting the first spring to unload. The jump in measured force may be due to the reaction force from the first spring. It is also noted that the third peak is lower than the first peak in the measured force. This difference in force may be due to vacuum generation. As the first spring unloads in this second phase of vacuum generation, the measured force gradually decreases. In the meantime, as the device moves through the second phase of vacuum generation, the buffer volume pressure continues to decrease, thus increasing the vacuum level in the buffer volume. The buffer volume pressure curve crosses the target pressure reference line near the end of the second phase of vacuum generation, indicating that the device has achieved the desired vacuum level.

It should be appreciated that the vacuum generation device 20 may be altered to have a different measured force and/or buffer volume pressure profile. For example, in some embodiments, the peak measured force in the second phase of priming may exceed the peak measured force in the first phase of priming.

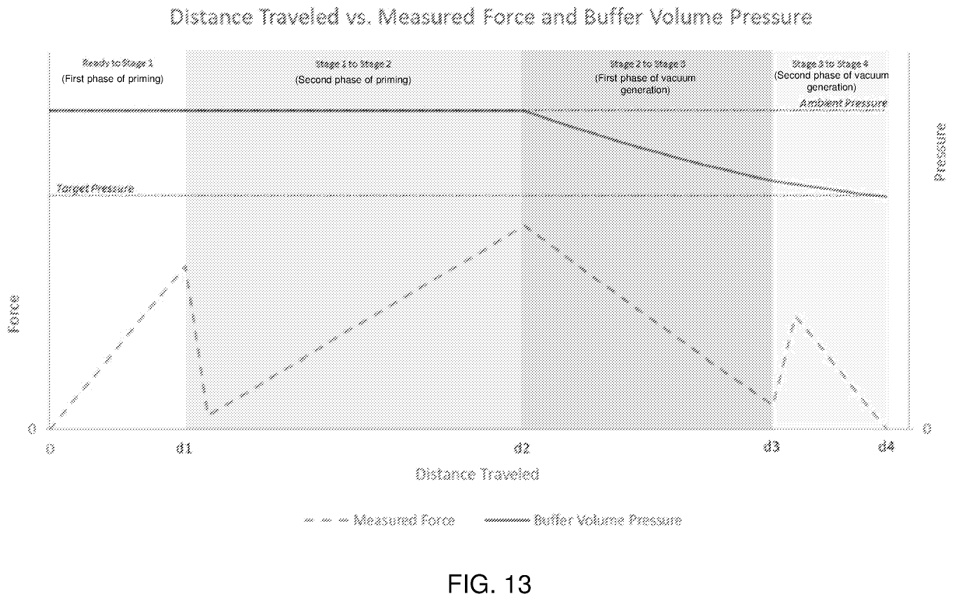

Such an arrangement is depicted in the graph of FIG. 13, which depicts a graph of measured force and buffer volume pressure profiles of a vacuum generation device through each operational stage.

The graph may, for example, represent the force and pressure profiles of the vacuum generation device 20 through each operational stage.

As seen in FIG. 13, the measured force reaches a higher magnitude in the second phase of priming than at the first phase of priming. This may occur, for example, due to using a certain stiffness with the second spring in combination with loading the second spring a certain distance. It is noted that the first spring loaded in the first phase of priming still has a higher stiffness than the second spring loaded in the second phase of priming, as reflected by the steeper slope of the measured force curve in the first phase of priming relative to the second phase of priming. However, loading a stiffer spring in the first phase of priming may not necessarily always result in a higher peak measured force in the first phase of priming, as shown in the graph of FIG. 13.

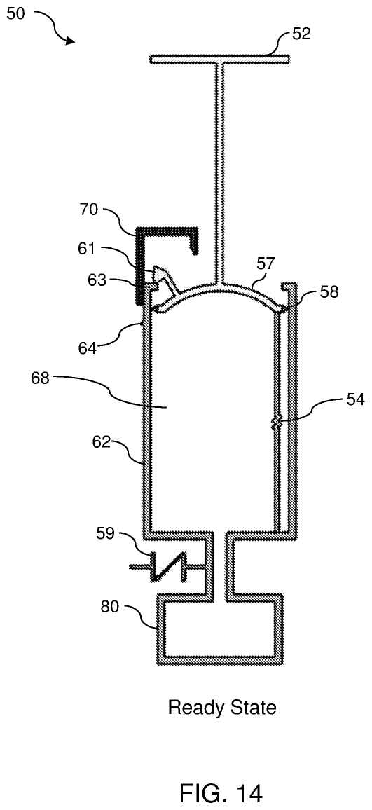

Another embodiment of a vacuum generation device is shown in FIG. 14. In the embodiment of FIG. 14, a bi-stable element 57 serves as a first return element, and a spring 54 serves as a second return element. The bi-stable element 57 comprises a bi-stable dome having a first stable state in which the dome is concave down, and a second stable state in which the dome inverts and is concave up. Once in the second stable state, the bi-stable dome does not spontaneously return to the first stable state. Instead, an applied force is required to move the bi-stable dome back to its first stable state. The bi-stable dome has a trip force, which is the actuation force required to invert the dome.

In FIG. 14, the vacuum generation device 50 is shown in the ready state. A user may manually prime the device by pushing on the actuator 52. According to one aspect, the priming step may have a first phase and a second phase. In some embodiments, the force required to actuate the actuator is higher in the first phase than in the second phase. Alternatively or in addition, in some embodiments, a first return element is loaded in the first phase and a second return element is loaded in the second phase, wherein a stiffness of the first return element is higher than a stiffness of the second return element.

In the embodiment of FIG. 14, during priming, the bi-stable element 57 and the spring 54 are loaded. The vacuum generation device 50 includes a locking arrangement that separates the loading of the bi-stable element 57 from the loading of the spring 54. The spring 54 may have a lower stiffness than that of the bi-stable element 57. The locking arrangement may be configured to permit the stiffer bi-stable element 57 to be loaded first during a first phase of a priming step, while prohibiting the less stiff spring 54 from being loaded. During a second phase of the priming step, the locking arrangement may permit the less stiff spring 54 to be loaded.

In the embodiment of FIG. 14, the locking arrangement that separates the loading of the return elements includes a trigger 61 and a slide latch 70. When force is initially applied to the actuator 52, force is transmitted to the trigger 61 and to the bi-stable element 57. The trigger 61, which is engaged with a ledge 63 of the housing 62, prevents compression of the spring 57 until the applied force is sufficient to invert the bi-stable element 57 to the second stable state. With a seal 58 sealing the bi-stable element against the housing 62, the bi-stable element acts as a piston. Inversion of the bi-stable element to the second state decreases the active volume 68 below the bi-stable element 57. As the active volume 68 decreases, pressure buildup is relieved via the one-way valve 59.

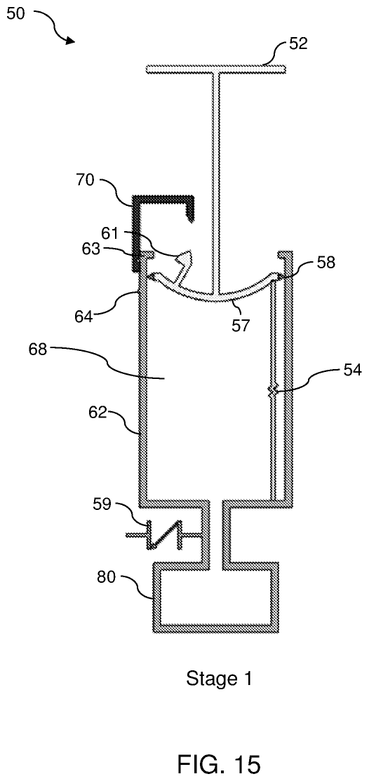

As shown in FIG. 15, which depicts the device 50 in stage 1, inversion of the bi-stable element 57 disengages the trigger 61 from the housing 62 and permits compression of the spring 54.

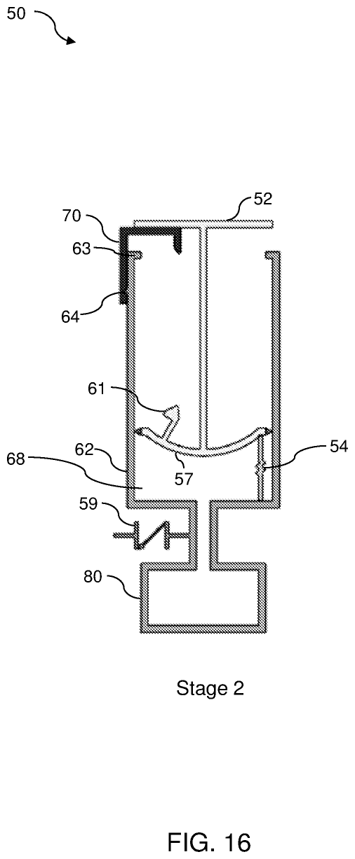

The user continues to press down on the actuator 52 to compress the spring 54 until reaching stage 2, depicted in FIG. 16. The active volume 68 has been reduced even further, with pressure buildup being relieved through the one-way valve 59. At stage 2, the actuator 52 engages with the slide latch 70, pushing the slide latch 70 downwards until the slide latch engages with a slide latch lock 64 on the housing 62. At stage 2, the priming step has ended.

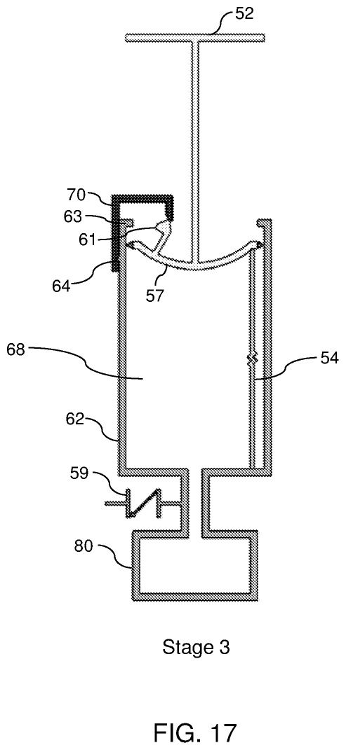

Next, the user ceases applying force to the actuator, transitioning the device to the vacuum generation step, which is stage 3, depicted in FIG. 17. With the removal of application of force on the actuator, the spring 54 decompresses, causing the bi-stable element 57 to begin to move back in the direction that increases the volume of the active volume 68 (upward in FIG. 17). As the one-way valve 59 does not permit entry of air into the active volume 68, vacuum is generated as the active volume 68 increases. As the bi-stable element 57 moves upward, the trigger 61 moves upward with the bi-stable element, causing the trigger to engage with the slide latch 70 to trigger inversion of the bi-stable element back to the first stable state (concave downward).

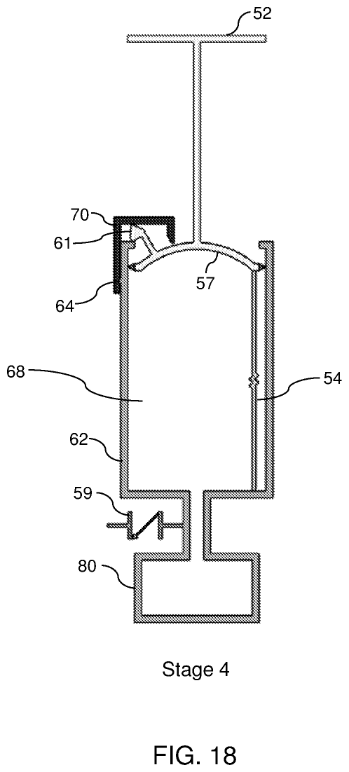

Inversion of the bi-stable element 57 back to the first stable state is shown in FIG. 18, which depicts the device in stage 4. Inversion of the bi-stable element 57 increases the active volume 68, which results in further vacuum generation at the buffer volume 80. As a result, the device has achieved a target vacuum level in the buffer volume 80.

A general aspect described above relates to the use of a locking arrangement that separates return elements such that the return elements are loaded independently during a priming step and unloaded independently during a vacuum generation step. While the illustrative embodiments shown in the figures each include a first return element and a second return element, it should be appreciated that any number of return elements may be used in a vacuum generation device. The devices shown in the drawings can be extrapolated to include more than two return elements. For example, a vacuum generation device may have four return elements and a locking arrangement that permits each return element to be loaded independently and unloaded independently. During a priming step, the locking arrangement may permit a first return element to be loaded while the other three return elements are locked from being loaded, and the locking arrangement may then transition to unlocking the second return element for loading while locking the other three from loading, and so on.

During a vacuum generation step, the locking arrangement may permit the fourth return element to be unloaded while the other three return elements are locked from being unloaded, and the locking arrangement may then transition to unlocking the third return element for unloading while locking the other three from unloading, and so on.

In some embodiments, the locking arrangement may be configured to unlock the return elements in a sequential order during a priming step, starting from the stiffest return element, to the next stiffest return element, and so on until finally unlocking the least stiff return element. Similarly, in a vacuum generation step, the locking arrangement may be configured to unlock the return elements in a reverse sequential order, starting from the least stiff return element and ending with the stiffest return element. In this manner, the device may utilize the return element with the greatest reaction force toward the end of the vacuum generation step, which may also be when the level of vacuum force tending to resist return is highest.

It should also be appreciated that any of the return elements may or may not have some initial load (e.g., a spring that is pre-loaded with an initial elongation or compression).

In the illustrative embodiments described above, the vacuum generation devices included different types of locking arrangements. In the embodiment of FIG. 5, the locking arrangement of the device 20 includes friction pads, a swing latch, and a ratchet lock. In the embodiment of FIG. 14, the locking arrangement of the device 50 includes a trigger and ledge, a slide latch, and a slide latch lock. It should be appreciated that other locking arrangements are possible. For example, locking arrangements may include other types of latches, including linear latches and rotary latches, electromechanical devices, electromagnets (e.g. including, but not limited to, solenoids), magnets, breakaway connections, or any other suitable arrangements. For example, a breakaway connection may be used to temporarily lock a second or subsequent return element from being loaded. The breakaway connection could, for example, substitute the friction pads of the embodiment of FIG. 5.

In some cases, devices such as the ones described herein can be used for receiving fluids or other materials, such as blood or interstitial fluid, from subjects, e.g., from the skin and/or beneath the skin. However, the devices described herein are not limited to only such applications. In other cases, devices such as are described herein may be used in any application where a vacuum is desired to be created. Examples include, for example, priming of oil pumps, creation of suctions to attach objects together, or movement of fluids or other substances from one location to another location.

It is appreciated that, with some fluid receiving devices, when obtaining fluid from pierced skin via vacuum, the device is pressed down onto the skin, which imparts a force onto the skin. It is recognized that the force imparted to the skin may have an effect on the quality and quantity of fluid that is withdrawn from the skin. For example, in the case of blood, it is recognized that, if the device pinches, compresses and/or stretches the skin too much, fluid may be impeded from being withdrawn from the skin, e.g. due to blood vessels deforming and collapsing in reaction to the forces imparted to the skin.

According to one aspect, in some embodiments, the device has an interface that helps distribute force to the skin to avoid high concentrations of force on the skin. It is also recognized that, in some embodiments, piercing into and withdrawing fluid from skin that has bulged into certain shapes in reaction to application of vacuum may give rise to increased fluid volume and/or quality. Improved quality may include factors such as avoiding damage to red blood cells and release of cell contents such as hemoglobin or potassium, or decreasing activation of coagulation. In some cases, a skin bulge may help piercing needles to penetrate into the skin at the full insertion depth of the needles. The skin bulge may keep the skin taut, and it may be easier for needles to penetrate into taut skin.

It is also recognized that, in some cases, bulging of skin may induce vasodilation and increase blood flow. For instance, a skin bulge may result in bulge tissue deformation that mechanically dilates blood vessels that reside within the tissue. This vasodilation could be amplified by the body's physiological response to the forces applied to the skin.

According to one aspect, in some embodiments, the device has an interface that permits skin movement under the device to permit skin recruitment into a device opening and to promote desirable skin bulging to facilitate piercing of skin and subsequent withdrawal of fluid from the skin. In some cases, skin movement might happen after piercing of skin, e.g., movement may occur due to vacuum that is generated after skin is pierced.

According to another aspect, in some embodiments, the device interface helps to maintain a seal with the skin during skin bulging and/or other movement.

Embodiments described herein relate to a fluid receiving device having an interface for facilitating fluid withdrawal from a subject. In some embodiments, the interface may be integrated with a fluid receiving module that may serve to promote withdrawal of fluid from a subject. The fluid receiving module may include one or more of the following components: a vacuum source, a fluid storage chamber, and a flow activator. In some embodiments, the fluid receiving device is arranged to pierce the skin of a subject, subject the pierced skin to vacuum to draw fluid out of the skin, and collect the fluid inside the device. The device may be arranged to deploy a plurality of microneedles into the skin. The device may be positioned on any suitable location on the subject, for example, on the arm or leg, on the back, on the abdomen, etc.

The subject is usually human, although non-human subjects may be used in certain instances, for instance, other mammals such as a dog, a cat, a horse, a rabbit, a cow, a pig, a sheep, a goat, a rat (e.g., Rattus norvegicus), a mouse (e.g., Mus musculus), a guinea pig, a hamster, a primate (e.g., a monkey, a chimpanzee, a baboon, an ape, a gorilla, etc.), or the like.

The device may be actuated by the subject, and/or by another person (e.g., a health care provider, such as a doctor), or the device itself may be self-actuating, e.g., upon application to the skin of a subject.

In one set of embodiments, the vacuum source is a pressure regulator that creates a pressure differential (such as a vacuum). The pressure regulator may be a pressure controller component or system able to create a pressure differential between two or more locations. The pressure differential should be at least sufficient to urge the movement of fluid or other material in accordance with various embodiments as discussed herein, and the absolute pressures at the two or more locations are not important so long as their differential is appropriate, and their absolute values are reasonable for the purposes discussed herein. For example, the pressure regulator may produce a pressure higher than atmospheric pressure in one location, relative to a lower pressure at another location (atmospheric pressure or some other pressure), where the differential between the pressures is sufficient to cause fluid transport. In another example, the regulator or controller will involve a pressure lower than atmospheric pressure (a vacuum) in one location, and a higher pressure at another location(s) (atmospheric pressure or a different pressure) where the differential between the pressures is sufficient to transport fluid. Wherever “vacuum” or “pressure” is used herein, in association with a pressure regulator or pressure differential, it should be understood that the opposite can be implemented as well, as would be understood by those of ordinary skill in the art, e.g., a vacuum chamber can be replaced in many instances with a pressure chamber, for creating a pressure differential suitable for causing the transport of fluid or other material.

In some embodiments, the vacuum source is a component that a user may actuate to generate a vacuum. Vacuum sources that are actuated to generate a vacuum may be purely mechanical, or may require electricity to operate (e.g. battery-operated or wired to receive electricity from a wall socket). In some embodiments, the vacuum source has a moveable component such as a flexible membrane, a piston, an expandable foam, or a shape memory material that is moved to generate a vacuum. In some illustrative embodiments described in further detail below, the vacuum source may be compressible and may be biased to return to an expanded state, generating vacuum from a compressed state to an expanded state. In some embodiments, the chamber does not include a flexible dome, e.g., one that is biased to return toward a non-compressed state. In another set of embodiments, however, the chamber does include such a dome.

In some embodiments, the vacuum source may be actuated a single time to generate sufficient vacuum. In other embodiments, the vacuum source may be repeatedly actuated (e.g. repeated pumping) to generate the desired vacuum.

In some embodiments, the vacuum source is a pre-packaged vacuum—a volume or chamber that has been pre-evacuated at manufacturing to be at a pressure that is less than ambient pressure. In some embodiments, a user may actuate the fluid receiving device to open fluid communication to the pre-packaged vacuum chamber. Thus, the device contains a “pre-packaged” vacuum chamber, such that it is received “ready for use,” without requiring any actuation to produce a vacuum within the vacuum chamber. In some embodiments, the vacuum source is a Vacutainer™ tube, a Vacuette™ tube, or other commercially-available vacuum tube.

In some embodiments, the device operation is entirely mechanical and does not require a power source (e.g. electrical, battery) or software electronics.

In some embodiments, the vacuum source may be a vacuum pump that is able to create a vacuum within the device. In some embodiments, the vacuum source may include chemicals or other reactants that can react to increase or decrease pressure which, with the assistance of mechanical or other means driven by the reaction, can form a pressure differential. In some embodiments, chemical reaction can drive mechanical actuation to form a pressure differential without a change in pressure based on the chemical reaction itself.

Other examples of a vacuum source include: a syringe pump, a piston pump, a syringe, a bulb, a Venturi tube, manual (mouth) suction. In some embodiments, a vacuum source comprises a spring-loaded mechanism. The user may cock the spring-loaded mechanism during use of the device. In other embodiments, the spring-loaded mechanism may be supplied pre-cocked prior to device actuation, and a user may release the mechanism by, e.g., actuating a device actuator.

In some embodiments, a vacuum may be created by a vacuum source without an external power and/or an external vacuum source, e.g., the vacuum source may be self-contained within the device. For instance, a vacuum may be created through a change in shape of a portion of the device (e.g., using a shape memory polymer). As a specific example, a shape memory polymer may be shaped to be flat at a first temperature (e.g., room temperature) but curved at a second temperature (e.g., body temperature), and when applied to the skin, the shape memory polymer may alter from a flat shape to a curved shape, thereby creating a vacuum. As another example, a mechanical device may be used to create the vacuum, for example, springs, coils, expanding foam (e.g., from a compressed state), a shape memory polymer, shape memory metal, or the like may be stored in a compressed or wound released upon application to a subject, then released (e.g., unwinding, uncompressing, etc.), to mechanically create the vacuum.

Non-limiting examples of shape-memory polymers and metals include Nitinol, compositions of oligo(epsilon-caprolactone)diol and crystallizable oligo(rho-dioxanone)diol, or compositions of oligo(epsilon-caprolactone)dimethacrylate and n-butyl acrylate.

In some embodiments, the device may include an indicator that provides an indication of the vacuum level that has been generated. In some embodiments, the indicator comprises a button or other element that retracts into the vacuum source (e.g. a vacuum chamber) due to being subjected by the vacuum generated by the vacuum source. In some embodiments, the indicator comprises a manometer or other pressure gauge.

According to one aspect, to promote withdrawal of fluid, the device includes an interface configured to contact skin, the interface being conformable to skin, e.g. the interface is able to deform to conform to skin as the skin bulges under vacuum. The interface may conform to the skin in an elastic (e.g. reversible) manner, or in a plastic (e.g. irreversible) manner.

In some embodiments, the interface may be conformable to skin due to the material that the interface is made from, and/or due to structural geometry of the device.

In some embodiments, the interface may be moveable relative to other components of the device, such as a rigid housing or a rigid support that connects the interface to other components of the device.

In some embodiments, the interface is made of a flexible material such as silicone, including ECOFLEX 10, ECOFLEX 30, DRAGONSKIN 30, SMOOTH-SIL 940, SMOOTH-SIL 950, and SMOOTH-SIL 960, each from SMOOTH-ON, INC. Any of these silicone materials may be combined with SLACKER (from SMOOTH-ON, INC.), a material that makes the silicone materials softer and tackier. In some embodiments, the interface is made of a thermoplastic elastomer, including thermoplastic vulcanizate. Examples of thermoplastic elastomers include, but are not limited to, SANTOPRENE 111-35, SANTOPRENE 211-35, SANTOPRENE 111-45, and SANTOPRENE 211-45 from EXXONMOBIL, or VERSAFLEX CL2242, VERSAFLEX CL2250, VERSAFLEX OM 1040X-1, and VERSAFLEX OM 1060X-1 from POLYONE. Examples of other possible flexible materials for the interface include, but are not limited to: polyurethanes, polystyrene/rubber block copolymers, e.g. Styrene-ethylene-butylene-styrene (SEBS), EPDM, and compressible foam (e.g., closed-cell foam or open-cell foam with a thin film coating to provide a seal against the skin).

In some embodiments, structural geometry of the device may permit the interface to be conformable to skin, (e.g., in some embodiments, moveable relative to other components of the device). For example, the device may include a region of decreased thickness that connects the interface to the rest of the device, and the decreased thickness may act as a flexure region, e.g. a hinge, that permits movement of the interface relative to other portions of the device. Other approaches may be used to achieve a flexure region, such as strategic removal of material (e.g. slits in the interface material), texturing of the material, co-molding of parts (e.g. a rigid outer material, a more flexible middle material, and a rigid inner material), forming the interface out of components(s) having non-uniform material properties, or a bellows design. In some embodiments, the interface may comprise a single part, or may comprise multiple parts. In some embodiments, the multiple parts may be moveable relative to one another, e.g. via a pivot relationship.

In some embodiments, an interface may be paired with a support that may connect the interface to other components of the device, such as a vacuum source or a device housing.

In some embodiments, the support may be more rigid than the interface. In some embodiments, the interface is made of a first material and the support is made of a second material, the first material having a lower Young's modulus than a Young's modulus of the second material. In other embodiments, however, the support and the interface may be made from the same material. In some of these embodiments, structural geometry of the device may permit the interface to be moveable relative to the support. For example, an area of decreased thickness or other shape that gives rise to a hinged arrangement may permit the interface to move relative to the support. In some embodiments, at least a portion of the interface may be thinner than at least a portion of the support.

The shape of the support may vary between different embodiments. In some embodiments, the support is a cylindrical shape with vertically straight walls. In some embodiments, the support is funnel-shaped, where the walls taper in a direction moving from the device opening into the device. In some cases, having a funnel-shaped support may help to distribute forces on the skin and/or may help to promote a desirable skin bulge. The support may have other shapes as well.

In some embodiments, the interface may be rigid rather than flexible. The rigid interface may be provided with one or more features to aid in some of the effects described above, such as promotion of skin recruitment to permit desirable skin bulging, as well as distribution of force on the skin. In some embodiments, a rigid interface may be coated with a lubricant to facilitate movement of skin under the interface. Examples of lubricant include, but are not limited to: petroleum jelly, glycerin, propylene glycol, hydroxyethylcellulose, hydroxypropylmethylcellulose, silicones, e.g. trisiloxane/dimethicone/cyclomethicone, fruit pectin, and extracts of aloe vera.

Materials for the rigid interface include, but are not limited to: photopolymerized methacrylic acid esters, polyethylene terephthalate (alcohol) esters (PET), polypropylene, polyethylene methyl acrylic ester, polycarbonate, polystyrene, poly ethylene, polyvinyl chloride, cycloolefin copolymer (COC), polytetrafluoroethylene, fluoropolymers, polyvinylidene chloride, polyimide, and polyester.

In some embodiments, the rigid interface may be shaped to have any of the same shapes as those discussed above for the support portion of the device.

It should be noted that a flow activator need not be included with all embodiments, as the device may not necessarily employ a mechanism for causing fluid release from the subject. For instance, the device may receive fluid that has already been released due to another cause, such as a cut or an abrasion, fluid release due to a separate and independent device, such as a separate lancet, an open fluid access such as during a surgical operation, and so on.

If included, a flow activator may physically penetrate, pierce, and/or or abrade, cut skin either laterally (e.g., slit) or rotationally (e.g., coring), chemically peel, corrode and/or irritate, release and/or produce electromagnetic, acoustic or other waves, other otherwise operate to cause fluid release from a subject. The flow activator may include a moveable mechanism, e.g., to move a needle, or may not require movement to function. For example, the flow activator may include a jet injector or a “hypospray” that delivers fluid under pressure to a subject, a pneumatic system that delivers and/or receives fluid, a hygroscopic agent that adsorbs or absorbs fluid, a reverse iontophoresis system, a transducer that emits ultrasonic waves, or thermal, radiofrequency and/or laser energy, and so on, any of which need not necessarily require movement of a flow activator to cause fluid release from a subject. In some embodiments, the flow activator may include one or more needles and/or blades.

It will be appreciated from the following description that the device may have needle deployment and retraction mechanisms that are conceptually similar in various aspects to the devices disclosed in International Application No. PCT/US2017/043580, filed Jul. 25, 2017, and U.S. Pat. No. 8,821,412, filed Nov. 19, 2012, the disclosures of which are incorporated by reference herein in their entireties.

In addition, the following documents are each incorporated herein by reference in their entities: Int Pat. Apl. Pub. Nos. WO 2010/101621, WO 2011/053787, WO 2011/053796, WO 2011/053788, WO 2011/094573, WO 2011/065972, WO 2011/088214, WO 2010/101626, WO 2011/163347, WO 2012/021792, WO 2012/021801, WO 2012/064802, WO 2011/088211, WO 2012/149143, WO 2012/149155, WO 2012/149126, WO 2012/154362, WO 2012/149134, WO 2016/123282, and WO 2018/022535.

In some cases, the device includes a chamber and a one-way vent. For instance, air may exit the chamber through the one-way vent during use, for instance, as the active volume of the chamber is decreased from the first volume to the second volume. Decreasing the volume of the chamber may initially create a pressure increase inside the device, but pressure will not build up inside the device due to the presence of a vent. Pressure may escape out through the vent. In some embodiments, the vent is a one-way vent. In some embodiments, the vent in the form of a valve. The valve may be a one-way valve such that airflow moves only from inside the device to the outside of the device, but not the other way around. For this and all other embodiments disclosed herein, examples of one-way valves include, but are not limited to, duckbill valves, ball check valves, umbrella valves, dome valves, Belleville valves, diaphragm check valves, swing check valves, stop-check valves, lift-check valves, in-line check valves, cross-slit valves, or any other suitable valve that allows fluid to pass in one direction only.

In some embodiments, the user's action may form part of a valve. For example, a gasket may be operably linked to a device actuator. Prior to device actuation, the gasket may be in a closed position. Actuation of the device actuator may cause the gasket to open and vent air.

It should be appreciated that a valve may not be necessary in all embodiments. For example, in some embodiments, the vacuum bulb may be pre-assembled within the device in a compressed state prior to device actuation.

In some embodiments, the chamber is biased toward returning to its original shape after application of force upon the vacuum bulb ceases. Thus, when a user stops depressing the device actuator, the chamber begins moving back to its original shape, thereby increasing the volume of space in the chamber. This volume increase creates a vacuum that promotes flow of fluid from the subject's pierced skin into the device.

The device may also include a storage chamber, e.g., for receiving fluid from the subject. In some embodiments, the storage container is in the form of a collection tube. The storage container may be sized and shaped for compatibility with other devices (which may, e.g. be commercially available from other parties), such as centrifuges, assay devices, or other analysis machines.

In some embodiments, the storage container contains one or more substances or objects prior to actuation of the device, and prior to entry of fluid from the subject into the storage container. For example, in some embodiments, the storage container may contain: sodium heparin, lithium heparin, balanced heparin, dipotassium EDTA, tripotassium EDTA, clot activator (such as silica), sodium citrate, sodium fluoride, sodium oxalate, acid citrate dextrose, a gel for separation during centrifugation, a mechanical barrier for separation during centrifugation, preservative for nucleic acids (DNA, RNA), any combination of the above, and any other suitable object or substance.

In some embodiments, a device may have two storage containers. In some embodiments, the device may require a user action to divert incoming substance(s) from the body to either the first storage container or the second storage container. In other embodiments, the device may automatically direct substances toward the first or second storage chamber.

As discussed above, in some embodiments, the device actuator may be a portion of the vacuum source, e.g., of the chamber described above.

According to one aspect, in some embodiments, a fluid receiving device may have a component that contacts and compresses the vacuum source rather than having the user contact the vacuum source (e.g., a chamber in the device, such as discussed herein) directly. It is appreciated that such a component may, in some embodiments, aid in compression of the vacuum source, e.g., may aid in complete, uniform, and/or consistent compression of the vacuum source.

In some embodiments, the component that contacts and compresses the vacuum source is attached to a device actuator that is distinct from the vacuum source. In some embodiments, the device actuator has a stem and a user-contacting portion, also referred to herein as a button. In some embodiments, the user-contacting portion and the stem are fixed to one another such that movement of one part moves the other, and vice versa. As a user presses down on the user-contacting portion, the stem may also contact and compress the vacuum source.

In some embodiments, the fluid receiving device includes a shell that constrains movement of the device actuator to aid in compression of the vacuum source by the device actuator. In some embodiments, the shell constrains movement of the device actuator to linear movement. The shell may be made of a material having a higher Young's modulus than the material of the vacuum source.

In some embodiments, the shell has an opening through which the device actuator moves. The stem of the device actuator may move through the opening of the shell. The opening and stem may be complementarily sized and shaped to constrain movement of the device actuator to linear movement.

In some embodiments, the fluid receiving device includes a ratchet mechanism that permits movement of the device actuator relative to the shell in one direction while resisting movement of the device actuator in the opposite direction. The ratchet mechanism may promote complete compression of the vacuum source by prohibiting the vacuum source from returning to its original shape until the vacuum source has been completely compressed. In some embodiments, the ratchet mechanism includes a ratchet located on the device actuator, and a pawl located on the shell, or vice versa. In some embodiments, the fluid receiving device includes a lockout that prohibits subsequent actuation of the device if the device has been previously actuated. In some embodiments, such a lockout may be provided by the ratchet mechanism. In some embodiments, the ratchet mechanism may be located on components other than, or in addition to, the device actuator.

According to one aspect, the device may include a piercing assembly that is configured to trigger release of needle(s) into skin in response to contact with skin. In some embodiments, the piercing assembly may be arranged in a floating arrangement in which a deployment actuator (e.g. a deployment spring) and needle assembly may move together as one unit in a deployment direction toward the device opening during device actuation. The deployment actuator may be triggered to deploy the needle assembly when a component of the piercing assembly has come into contact with skin. A skin contact-actuated arrangement may help to facilitate successful piercing of the user's skin and prevent premature activation of the device. In some embodiments, skin contact actuation may help to promote consistency of needle insertion across users having different skin characteristics (e.g. users having more compliant versus less compliant skin).

In some embodiments, the device includes a deployment actuator that moves the needles in a deployment direction toward the opening, and a retraction actuator that moves the needles in the opposite direction: a retraction direction away from the opening. In some embodiments, each of the deployment actuator and the retraction actuator act as springs that can be manipulated to store potential energy, and release of the stored potential energy drives movement of the needles.

It should be appreciated that the device may be actuated by various different actions/gestures. In some embodiments, the device may be actuated by squeezing, twisting, pulling, pressing, pinching, spinning, or by any other suitable action.

In some embodiments, a user may pull back on a spring-loaded rod and release, or otherwise actuate the device to cause a spring-loaded rod to be pulled back and released, causing needles to deploy into skin. In some embodiments, a user could cock a deployment mechanism to place a device in a ready to actuate state. E.g., a user could compress or pull back on a spring until it reaches a latched state, and then actuate a device actuator to release the cocked spring. In other embodiments, the spring may be assembled in a pre-compressed or pre-elongated state prior to any user interaction with the device, and the user may release the spring by, e.g., actuating a device actuator.

In some embodiments, the deployment actuator and the retraction actuator are springs. However, it should be appreciated that other arrangements for the deployment actuator and/or the retraction actuator are possible. For example, the deployment actuator and the retraction actuator may each include any number of suitable components, such as a button, a switch, a lever, a slider, a dial, a compression spring, a Belleville spring, compressible foam, a snap dome, a servo, rotary or linear electric motor, and/or a pneumatic apparatus, or other suitable device. Also, the deployment actuator and the retraction actuator may be of the same type, or may be different types of devices. Each actuator may operate manually, mechanically, electrically, pneumatically, electromagnetically, or other suitable mode of operation, and may or may not require user input for activation.

In some embodiments, the device may include an adhesive layer attached to the device interface that is configured to affix the interface to the surface of the skin. The adhesive layer may help to form a seal between the device interface and the skin, which may promote transfer of fluid from the body into the device (and/or transfer of substances from the device into the body).

As discussed above, in some embodiments, a device interface is made of a flexible material. The inventors have appreciated the technical challenges associated with attaching an adhesive layer onto a flexible material or other low surface energy material.

In one illustrative embodiment, an adhesive layer is heat-staked onto the device interface. In some embodiments, an adhesive layer that is heat-staked to the device interface may form a seal between the adhesive layer and the device interface. In some embodiments, the heat-staked adhesive layer may be able to withstand certain sterilization processes, such as gamma sterilization.

In some embodiments, the adhesive layer is a single-sided adhesive that includes an adhesive side and a non-adhesive backer side. In some embodiments, the process of heat-staking the adhesive layer to the device interface melts the backer to the device interface, thereby attaching the adhesive layer to the device interface.

In some embodiments, including some embodiments in which the adhesive is heat-staked to the device, the material of the backer is plastic. However, the backer is not necessarily limited to plastic in all embodiments. The backer may be made of any suitable material, including wovens and non-wovens, plastic, polyphenylene ether, elastomer, elastic polymer blend nonwoven, fiber-reinforced adhesive transfer tape, knit polyester tricot fabric, polyethylene, low density polyethylene, nylon, polyvinyl chloride foam, polyester, polyester spunlace, polyethylene/ethylene vinyl acetate, polyolefin, polyolefin foam, polyolefin foam covered wires, polypropylene, urethane, polyurethane, rayon nonwoven, rayon woven fabric, spunlace nonwoven, or thermoplastic elastomer film.

In some embodiments, including some embodiments in which adhesive is heat-staked to the device, the skin-side adhesive is made of an acrylate material. However, the adhesive is not necessarily limited to acrylate in all embodiments. The skin-side adhesive may be made of any suitable material, including acrylate, hydrocolloid, acrylic-based adhesives, silicone-based adhesives, hydrogel, pressure-sensitive adhesives, a contact adhesive, a permanent adhesive, a cyanoacrylate, glue, gum, hot melts, epoxy, or the like. In some cases, the adhesive is chosen to be biocompatible or hypoallergenic.

In some embodiments, the entire surface area of the interface bottom may be covered with an adhesive layer. In other embodiments, only a portion of the surface area of the interface bottom is covered with an adhesive layer.

In some embodiments, the adhesive-side of the adhesive layer may be initially covered with a liner that a user peels off to expose the adhesive prior to use of the device.

In another set of embodiments, the device may be mechanically held to the skin, for example, the device may include mechanical elements such as straps, belts, buckles, strings, ties, elastic bands, or the like. For example, a strap may be worn around the device to hold the device in place against the skin of the subject. In yet another set of embodiments, a combination of these and/or other techniques may be used. As one non-limiting example, the device may be affixed to a subject's arm or leg using adhesive and a strap.

In illustrative embodiments discussed above, the needle deployment and retraction mechanisms are used in devices in which vacuum is applied to the skin after needle insertion in to the skin. However, it should be appreciated that the needle deployment and retraction mechanisms described above may also be used in devices in which vacuum is applied to the skin before needle insertion into the skin. As an example, a pre-evacuated volume of space may be used as the vacuum source, in which case the device is configured to release vacuum from the pre-evacuated space prior to deployment of the needles.

In some embodiments, a deformable structure may serve as a needle deployment and/or retraction mechanism. In one set of embodiments, the deformable structure may move from a first position to a second position, and optionally the deformable structure may be able to reversibly move from the second position to the first position, i.e., the deformable structure may be a reversibly deformable structure. In some cases, the first position is stable and the second position is unstable, although in other cases both the first position and the second position are each stable (i.e., the reversibly deformable structure is bi-stable). In such a stable position, no external forces are needed to maintain equilibrium, i.e., its position.

For example, the first position may be one where the deformable structure is positioned such that the needle(s) do not contact the skin, while the second position may be one where the needle(s) contact the skin, and in some cases, the needle(s) may pierce the skin. The deformable structure may be moved using any suitable technique, e.g., manually, mechanically, electromagnetically, using a servo mechanism, or the like. In one set of embodiments, for example, the deformable structure may be moved from a first position to a second position by pushing a button, which causes the deformable structure to move (either directly, or through a mechanism linking the button with the support structure). Other mechanisms (e.g., dials, levers, sliders, etc., as discussed herein) may be used in conjunction of or instead of a button. In another set of embodiments, the deformable structure may be moved from a first position to a second position automatically, for example, upon activation by a computer, upon remote activation, after a period of time has elapsed, or the like. For example, in one embodiment, a servo connected to the deformable structure is activated electronically, moving the deformable structure from the first position to the second position.

In some cases, the deformable structure may also be moved from the second position to the first position. For example, after fluid has been delivered to and/or withdrawn from the skin and/or beneath the skin, e.g., using needle(s), the deformable structure may be moved, which may move the needle(s) away from contact with the skin. The deformable structure may be moved from the second position to the first position using any suitable technique, including those described above, and the technique for moving the support structure from the second position to the first position may be the same or different as that moving the support structure from the first position to the second position. In some cases, the deformable structure is reversibly deformable, i.e., the deformable structure is able to return from the second position back to the first position.

In one set of embodiments, the device includes a deformable structure able to drive needle(s) into the skin, e.g., so that the needle(s) can withdraw a fluid from the skin and/or from beneath the skin of a subject, and/or so that the needle(s) can deliver fluid or other material to a subject, e.g. deliver the fluid or other material to the skin and/or to a location beneath the skin of a subject. The deformable structure may be a structure that can be deformed using unaided force (e.g., by a human pushing the structure), or other forces (e.g., electrically-applied forces, mechanical interactions or the like), but is able to restore its original shape after the force is removed or at least partially reduced. For example, the structure may restore its original shape spontaneously, or some action (e.g., heating) may be needed to restore the structure to its original shape.

The deformable structure may be formed out of a suitable elastic material, in some cases. For example, the structure may be formed from a plastic, a polymer, a metal, etc. In one set of embodiments, the structure may have a concave or convex shape. For instance, the edges of the structure may be put under compressive stress such that the structure “bows” out to form a concave or convex shape. A person pushing against the concave or convex shape may deform the structure, but after the person stops pushing on the structure, the structure may be able to return to its original concave or convex shape, e.g., spontaneously or with the aid of other forces as previously discussed. In some cases, the device may be bi-stable, i.e., having two different positions in which the device is stable.

In one set of embodiments, the device may include a deformable structure that is moveable between a first configuration and a second configuration. For instance, the first configuration may have a concave shape, such as a dome shape, and the second configuration may have a different shape, for example, a deformed shape (e.g., a “squashed dome”), a convex shape, an inverted concave shape, or the like. The deformable structure may be moved between the first configuration and the second configuration manually, e.g., by pushing on the flexible concave member using a hand or a finger, and/or the deformable structure may be moved using an actuator such as is described herein. In some cases, the deformable structure may be able to spontaneously return from the second configuration back to the first configuration. In other cases, however, the deformable structure may not be able to return to the first configuration, for instance, in order to prevent accidental repeated uses of the deformable structure. The deformable structure, in some embodiments, may be a reversibly deformable structure, although in other embodiments, it need not be. In addition, in some cases, although the deformable structure may (or may not) be a reversibly deformable structure, the deformable structure may be moved from a first position to a second position using a first mechanism, and moved from the second position to the first position, or to a third position, using a second mechanism different from the first mechanism.

The deformable structure may be mechanically coupled to one or more needles (e.g., microneedles). The needle may be directly immobilized on the deformable structure, or the needles can be mechanically coupled to the deformable structure using bars, rods, levers, plates, springs, or other suitable structures. The needle(s), in some embodiments, are mechanically coupled to the deformable structure such that the needle is in a first position when the deformable structure is in a first configuration and the needle is in a second position when the deformable structure is in a second configuration.

In some cases, relatively high speeds and/or accelerations may be achieved, and/or insertion of the needle may occur in a relatively short period of time, e.g., as is discussed herein. The first position and the second position, in some cases, may be separated by relatively small distances. For example, the first position and the second position may be separated by a distance of less than about 10 mm, less than about 9 mm, less than about 8 mm, less than about 7 mm, less than about 6 mm, less than about 5 mm, less than about 4 mm, less than about 3 mm, or less than about 2 mm, etc. However, even within such distances, in certain embodiments, high speeds and/or accelerations such as those discussed herein can be achieved.

During use, a device may be placed into contact with the skin of a subject such that a recess or other suitable applicator region is proximate or in contact with the skin. By moving the deformable structure between a first configuration and a second configuration, because of the mechanical coupling, the deformable structure is able to cause a needle to move to a second position within the recess or other applicator region and to contact or penetrate the skin of the subject.

In some embodiments, the device may also include a retraction mechanism able to move the needle away from the skin after the deformable structure reaches a second configuration. Retraction of the deformable structure may, in some embodiments, be caused by the deformable structure itself, e.g., spontaneously returning from the second configuration back to the first configuration, and/or the device may include a separate retraction mechanism, for example, a spring, an elastic member, a collapsible foam, or the like. In other cases, however, a different mechanism may be used to retract the deformable structure. For example, the deformable structure may be in a second configuration, and withdrawn from the skin, e.g., laterally, without altering the configuration of the deformable structure.

The deformable structure may be formed from any suitable material, for example, a metal such as stainless steel (e.g., 301, 301LN, 304, 304L, 304LN, 304H, 305, 312, 321, 321H, 316, 316L, 316LN, 316Ti, 317L, 409, 410, 430, 440A, 440B, 440C, 440F, 904L), carbon steel, spring steel, spring brass, phosphor bronze, beryllium copper, titanium, titanium alloy steels, chrome vanadium, nickel alloy steels (e.g., Monel 400, Monel K 500, Inconel 600, Inconel 718, Inconel x 750, etc.), a polymer (e.g., polyvinylchloride, polypropylene, polycarbonate, etc.), a composite or a laminate (e.g., comprising fiberglass, carbon fiber, bamboo, Kevlar, etc.), or the like.

The deformable structure may be of any shape and/or size. In one set of embodiments, the deformable structure is not planar, and has a portion that can be in a first position (a “cocked” or predeployed position) or a second position (a “fired” or deployed position), optionally separated by a relatively high energy configuration. In some cases, both the first position and the second position are stable (i.e., the structure is bi-stable), although conversion between the first position and the second position requires the structure to proceed through an unstable configuration.

In one embodiment, the deformable structure is a flexible concave member. The deformable structure may have, for instance, a generally domed shape (e.g., as in a snap dome), and be circular (no legs), or the deformable structure may have other shapes, e.g., oblong, triangular (3 legs), square (4 legs), pentagonal (5 legs), hexagonal (6 legs), spider-legged, star-like, clover-shaped (with any number of lobes, e.g., 2, 3, 4, 5, etc.), or the like. The deformable structure may have, in some embodiments, a hole, dimple, or button in the middle. The deformable structure may also have a serrated disc or a wave shape. In some cases, the needle(s) may be mounted on the deformable structure. In other cases, however, the needle(s) are mounted on a separate structure which is driven or actuated upon movement of the deformable structure.

As used herein, “vacuum” generally refers to an amount of pressure below atmospheric pressure, such that atmospheric pressure has a vacuum of 0 mmHg, i.e., the pressure is gauge pressure rather than absolute pressure. For example, a vacuum may have a pressure of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least about 550 mmHg, at least about 600 mmHg, at least about 650 mmHg, at least about 700 mmHg, or at least about 750 mmHg below atmospheric pressure, i.e., a pressure that is reduced, as compared to standard atmospheric pressure. For instance, a vacuum pressure of 100 mmHg corresponds to an absolute pressure of about 660 mmHg (i.e., 100 mmHg below 1 atm).

The vacuum may be applied to any suitable region of the skin, and the area of the skin to which the vacuum may be controlled in some cases. For instance, the average diameter of the region to which vacuum is applied may be kept to less than about 5 cm, less than about 4 cm, less than about 3 cm, less than about 2 cm, less than about 1 cm, less than about 5 mm, less than about 4 mm, less than about 3 mm, less than about 2 mm, or less than about 1 mm. In addition, such vacuums may be applied for any suitable length of time. For instance, vacuum may be applied to the skin for at least about 1 min, at least about 3 min, at least about 5 min, at least about 10 min, at least about 15 min, at least about 30 min, at least about 45 min, at least about 1 hour, at least about 2 hours, at least about 3 hours, at least about 4 hours, etc. Different amounts of vacuum may be applied to different subjects in some cases, for example, due to differences in the physical characteristics of the skin of the subjects.

In some embodiments, the flow activator may include one or more needles and/or blades. In some embodiments, the needle(s) is(are) a microneedle(s). The needles may be arranged in a variety of different ways, depending on the intended application. For example, in some embodiments, the needle(s) may have a length of less than or equal to about 5 mm, less than or equal to about 4 mm, less than or equal to about 3 mm, less than or equal to about 2 mm, less than or equal to about 1 mm, less than or equal to about 800 micrometers, less than or equal to 600 micrometers, less than or equal to about 500 micrometers, less than or equal to about 400 micrometers, less than or equal to about 300 micrometers, less than or equal to about 200 micrometers, less than or equal to about 175 micrometers, less than or equal to about 150 micrometers, less than or equal to about 125 micrometers, less than or equal to about 100 micrometers, less than or equal to about 75 micrometers, less than or equal to about 50 micrometers, less than or equal to about 10 micrometers, etc.

In some embodiments, the needle(s) may have a largest cross-sectional dimension of less than or equal to about 5 mm, less than or equal to about 4 mm, less than or equal to about 3 mm, less than or equal to about 2 mm, less than or equal to about 1 mm, less than or equal to about 800 micrometers, less than or equal to 600 micrometers, less than or equal to 500 micrometers, less than or equal to 400 micrometers, less than or equal to about 350 micrometers, less than or equal to about 300 micrometers, less than or equal to about 200 micrometers, less than or equal to about 175 micrometers, less than or equal to about 150 micrometers, less than or equal to about 125 micrometers, less than or equal to about 100 micrometers, less than or equal to about 75 micrometers, less than or equal to about 50 micrometers, less than or equal to about 10 micrometers, etc.

In some embodiments, the largest cross-sectional dimension of the needle is the width of the needle. In some embodiments, the largest cross-sectional dimension of the needle is the thickness of the needle. In some embodiments, the largest cross-sectional dimension of the needle is the diameter of the needle. Depending upon the geometry of the needle, some or all of these terms (i.e. width, thickness, diameter) may be interchangeable.

For example, some embodiments include needles having a rectangular cross-section, and may have a thickness and a width that are distinct from one another. Some embodiments include needles having a circular cross-section, where its largest cross-sectional dimension of the needle is the diameter of the circular cross-section.

In some embodiments, the needle(s) may have a rectangular cross section having dimensions of 175 micrometers by 50 micrometers, or 350 micrometers by 50 micrometers.

In one set of embodiments, the needle(s) may have an aspect ratio of length to largest cross-sectional dimension of at least about 2:1, at least about 3:1, at least about 4:1, at least 5:1, at least about 7:1, at least about 10:1, at least about 15:1, at least about 20:1, at least about 25:1, at least about 30:1, etc.

It should be understood that references to “needle” or “microneedle” as discussed herein are by way of example and ease of presentation only, and that in other embodiments, more than one needle and/or microneedle may be present in any of the descriptions herein.

As an example, microneedles such as those disclosed in U.S. Pat. No. 6,334,856, issued Jan. 1, 2002, entitled “Microneedle Devices and Methods of Manufacture and Use Thereof,” by Allen, et al., may be used to deliver to and/or withdraw fluids (or other materials) from a subject. The microneedles may be hollow or solid, and may be formed from any suitable material, e.g., metals, ceramics, semiconductors, organics, polymers, and/or composites. Examples include, but are not limited to, medical grade stainless steel, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, polymethyl methacrylate, polyacrylic acid, or polyesters.

In some cases, more than one needle or microneedle may be used. For example, arrays of needles or microneedles may be used, and the needles or microneedles may be arranged in the array in any suitable configuration, e.g., periodic, random, etc. In some cases, the array may have 3 or more, 4 or more, 5 or more, 6 or more, 10 or more, 15 or more, 20 or more, 35 or more, 50 or more, 100 or more, or any other suitable number of needles or microneedles. Typically, a microneedle will have an average cross-sectional dimension (e.g., diameter) of less than about a micron.

In one illustrative embodiment, the flow activator includes an array of microneedles that are arranged in a 7.5 mm diameter circular pattern with 30 microneedles around the circumference. Each of the microneedles is 1 mm long and 0.350 mm wide.

Those of ordinary skill in the art can arrange needles relative to the skin or other surface for these purposes including, in one embodiment, introducing needles into the skin at an angle, relative to the skin's surface, other than 90 degrees, i.e., to introduce a needle or needles into the skin in a slanting fashion so as to limit the depth of penetration. In another embodiment, however, the needles may enter the skin or other surface at approximately 90 degrees.

In some cases, the needles (or microneedles) may be present in an array selected such that the density of needles within the array is between about 0.5 needles/mm² and about 10 needles/mm², and in some cases, the density may be between about 0.6 needles/mm² and about 5 needles/mm², between about 0.8 needles/mm² and about 3 needles/mm², between about 1 needles/mm² and about 2.5 needles/mm², or the like. In some cases, the needles may be positioned within the array such that no two needles are closer than about 1 mm, about 0.9 mm, about 0.8 mm, about 0.7 mm, about 0.6 mm, about 0.5 mm, about 0.4 mm, about 0.3 mm, about 0.2 mm, about 0.1 mm, about 0.05 mm, about 0.03 mm, about 0.01 mm, etc.

In another set of embodiments, the needles (or microneedles) may be chosen such that the area of the needles (determined by determining the area of penetration or perforation on the surface of the skin of the subject by the needles) allows for adequate flow of fluid to or from the skin and/or beneath the skin of the subject. The needles may be chosen to have smaller or larger areas (or smaller or large diameters), so long as the area of contact for the needles to the skin is sufficient to allow adequate blood flow from the skin of the subject to the device. For example, in certain embodiments, the needles may be selected to have a combined skin-penetration area of at least about 500 nm², at least about 1,000 nm², at least about 3,000 nm², at least about 10,000 nm², at least about 30,000 nm², at least about 100,000 nm², at least about 300,000 nm², at least about 1 microns², at least about 3 microns², at least about 10 microns², at least about 30 microns², at least about 100 microns², at least about 300 microns², at least about 500 microns², at least about 1,000 microns², at least about 2,000 microns², at least about 2,500 microns², at least about 3,000 microns², at least about 5,000 microns², at least about 8,000 microns², at least about 10,000 microns², at least about 35,000 microns², at least about 100,000 microns², at least about 300,000 microns², at least about 500,000 microns², at least about 800,000 microns², at least about 8,000,000 microns², etc., depending on the application.

The needles or microneedles may have any suitable length, and the length may be, in some cases, dependent on the application. For example, needles designed to only penetrate the epidermis may be shorter than needles designed to also penetrate the dermis, or to extend beneath the dermis or the skin. In certain embodiments, the needles or microneedles may have a maximum penetration into the skin of no more than about 3 mm, no more than about 2 mm, no more than about 1.75 mm, no more than about 1.5 mm, no more than about 1.25 mm, no more than about 1 mm, no more than about 900 microns, no more than about 800 microns, no more than about 750 microns, no more than about 600 microns, no more than about 500 microns, no more than about 400 microns, no more than about 300 microns, no more than about 200 microns, no more than about 175 micrometers, no more than about 150 micrometers, no more than about 125 micrometers, no more than about 100 micrometers, no more than about 75 micrometers, no more than about 50 micrometers, etc. In certain embodiments, the needles or microneedles may be selected so as to have a maximum penetration into the skin of at least about 50 micrometers, at least about 100 micrometers, at least about 300 micrometers, at least about 500 micrometers, at least about 1 mm, at least about 2 mm, at least about 3 mm, etc.

In one set of embodiments, the needles (or microneedles) may be coated. For example, the needles may be coated with a substance that is delivered when the needles are inserted into the skin. For instance, the coating may comprise heparin, an anticoagulant, an anti-inflammatory compound, an analgesic, an anti-histamine compound, etc. to assist with the flow of blood from the skin of the subject, or the coating may comprise a drug or other therapeutic agent such as those described herein. The drug or other therapeutic agent may be one used for localized delivery (e.g., of or proximate the region to which the coated needles or microneedles are applied), and/or the drug or other therapeutic agent may be one intended for systemic delivery within the subject.

In some cases, at least a portion of the fluid received by the device from the subject may be stored, and/or analyzed to determine one or more analytes, e.g., a marker for a disease state, or the like. The fluid withdrawn from the subject may be subjected to such uses. The fluid may be removed using any suitable technique, e.g., as discussed herein.

Thus, the device, in one set of embodiments, may involve determination of a condition of a subject. A variety of sensors may be used, many of which are commercially readily available. In such a case, a bodily fluid received by the device may be analyzed, for instance, as an indication of a past, present and/or future condition of the subject, or to determine conditions that are external to the subject. The condition of the subject to be determined may be one that is currently existing in the subject, and/or one that is not currently existing, but the subject is susceptible or otherwise is at an increased risk to that condition. The condition may be a medical condition, e.g., diabetes or cancer, or other physiological conditions, such as dehydration, pregnancy, illicit drug use, or the like.

Additional non-limiting examples are discussed herein. Determination may occur, for instance, visually, tactilely, by odor, via instrumentation, etc.

In some cases, such fluids will contain various analytes within the body that are important for diagnostic purposes, for example, markers for various disease states, such as glucose (e.g., for diabetics); other example analytes include ions such as sodium, potassium, chloride, calcium, magnesium, and/or bicarbonate (e.g., to determine dehydration); gases such as carbon dioxide or oxygen; H⁺ (i.e., pH); metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Other non-limiting examples include insulin or hormones.

For instance, fluids withdrawn from the skin of the subject will often contain various analytes within the body that are important for diagnostic purposes, for example, markers for various disease states, such as glucose (e.g., for diabetics); other example analytes include ions such as sodium, potassium, chloride, calcium, magnesium, and/or bicarbonate (e.g., to determine dehydration); gases such as carbon dioxide or oxygen; H⁺ (i.e., pH); metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Other examples include insulin, or hormone levels. Still other analytes include, but not limited to, high-density lipoprotein (“HDL”), low-density lipoprotein (“LDL”), albumin, alanine transaminase (“ALT”), aspartate transaminase (“AST”), alkaline phosphatase (“ALP”), bilirubin, lactate dehydrogenase, etc. (e.g., for liver function tests); luteinizing hormone or beta-human chorionic gonadotrophin (hCG) (e.g., for fertility tests); prothrombin (e.g., for coagulation tests); troponin, BNT or B-type natriuretic peptide, etc., (e.g., as cardiac markers); infectious disease markers for the flu, respiratory syncytial virus or RSV, etc.; or the like.

Other conditions that can be determined by the device include pH or metal ions, proteins, enzymes, antibodies, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. Other conditions that can be determined include pH changes, which may indicate disease, yeast infection, periodontal disease at a mucosal surface, oxygen or carbon monoxide levels which indicate lung dysfunction, and drug levels, both legal prescription levels of drugs such as coumadin and illegal such as cocaine or nicotine. Further examples of analytes include those indicative of disease, such as cancer specific markers such as CEA and PSA, viral and bacterial antigens, and autoimmune indicators such as antibodies to double stranded DNA, indicative of Lupus. Still other conditions include exposure to elevated carbon monoxide, which could be from an external source or due to sleep apnea, too much heat (important in the case of babies whose internal temperature controls are not fully self-regulating) or from fever. Still other potentially suitable analytes include various pathogens such as bacteria or viruses (for example, coronaviruses such as SARS-CoV-2), and/or markers produced by such pathogens. Thus, in certain embodiments, one or more analytes within the skin or within the body may be determined in some fashion, which may be useful in determining a past, present and/or future condition of the subject.

In some cases, fluids or other materials received from the subject may be used to determine conditions that are external to the subject. For example, the fluids or other materials may contain reaction entities able to recognize pathogens or other environmental conditions surrounding the subject, for example, an antibody able to recognize an external pathogen (or pathogen marker). As a specific example, the pathogen may be anthrax and the antibody may be an antibody to anthrax spores. As another example, the pathogen may be a Plasmodia (some species of which causes malaria) and the antibody may be an antibody that recognizes the Plasmodia. As yet another example, the pathogen may be a virus, such as a coronavirus (e.g., SARS-CoV-2), and the antibody may be an antibody able to bind to at least a portion of the virus, such as a spike protein, an envelope protein, a membrane protein, etc.

In some embodiments, upon determination of the fluid and/or an analyte present or suspected to be present within the fluid, a microprocessor or other controller may display a suitable signal on a display. The display may also be used to display other information, in addition or instead of the above. For example, the device may include one or more displays that indicate when the device has been used or has been expired, that indicate that sampling of fluid from a subject is ongoing and/or complete, or that a problem has occurred with sampling (e.g., clogging or insufficient fluid collected), that indicate that analysis of an analyte within the collected sample is ongoing and/or complete, that an adequate amount of a fluid has been delivered to the subject (or that an inadequate amount has been delivered, and/or that fluid delivery is ongoing), that the device can be removed from the skin of the subject (e.g., upon completion of delivery and/or withdrawal of a fluid, and/or upon suitable analysis, transmission, etc.), or the like.

However, a display is not a requirement; in other embodiments, no display may be present, or other signals may be used, for example, lights, smell, sound, feel, taste, or the like. Any of a variety of signaling or display methods, associated with analyses, can be provided including signaling visually, by smell, sound, feel, taste, or the like, in one set of embodiments. Signal structures and generators include, but are not limited to, displays (visual, LED, light, etc.), speakers, chemical-releasing chambers (e.g., containing a volatile chemical), mechanical devices, heaters, coolers, or the like. In some cases, the signal structure or generator may be integral with the device (e.g., integrally connected with a support structure for application to the skin of the subject, e.g., containing a fluid transporter such as a needle or a microneedle), or the signal structure or generator may not be integrally connected with the support structure.

In some cases, the device may contain a sensor for determining a fluid and/or an analyte within the fluid. In certain embodiments, the device may contain reagents able to interact with an analyte contained or suspected to be present within the fluid from the subject, for example, a marker for a disease state. As non-limiting examples, the sensor may contain an antibody able to interact with a marker for a disease state, an enzyme such as glucose oxidase or glucose 1-dehydrogenase able to detect glucose, or the like. The analyte may be determined quantitatively or qualitatively, and/or the presence or absence of the analyte within the withdrawn fluid may be determined in some cases.

Additional non-limiting examples of sensors include, but are not limited to, pH sensors, optical sensors, ion sensors, colorimetric sensors, a sensor able to detect the concentration of a substance, or the like, e.g., as discussed herein. For instance, in one set of embodiments, the device may include an ion selective electrode. The ion selective electrode may be able to determine a specific ion and/or ions such as K⁺, H⁺, Na⁺, Ag⁺, Pb²⁺, Cd²⁺, or the like. Various ion selective electrodes can be obtained commercially. As a non-limiting example, a potassium-selective electrode may include an ion exchange resin membrane, using valinomycin, a potassium channel, as the ion carrier in the membrane to provide potassium specificity. Those of ordinary skill in the art will be aware of many suitable commercially-available sensors, and the specific sensor used may depend on the particular analyte being sensed.

The sensor may be, for example, embedded within or integrally connected to the device, or positioned remotely but with physical, electrical, and/or optical connection with the device so as to be able to sense a chamber within the device. For example, the sensor may be in fluidic communication with fluid withdrawn from a subject, directly, via a microfluidic channel, an analytical chamber, etc. The sensor may be able to sense an analyte, e.g., one that is suspected of being in a fluid withdrawn from a subject. For example, a sensor may be free of any physical connection with the device, but may be positioned so as to detect the results of interaction of electromagnetic radiation, such as infrared, ultraviolet, or visible light, which has been directed toward a portion of the device, e.g., a chamber within the device. As another example, a sensor may be positioned on or within the device, and may sense activity in a chamber by being connected optically to the chamber. Sensing communication can also be provided where the chamber is in communication with a sensor fluidly, optically or visually, thermally, pneumatically, electronically, or the like, so as to be able to sense a condition of the chamber. As one example, the sensor may be positioned downstream of a chamber, within a channel such a microfluidic channel, or the like.

The sensor may be, for example, a pH sensor, an optical sensor, an oxygen sensor, a sensor able to detect the concentration of a substance, or the like. Other examples of analytes that the sensor may be used to determine include, but are not limited to, metal ions, proteins, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. Non-limiting examples of sensors include dye-based detection systems, affinity-based detection systems, microfabricated gravimetric analyzers, CCD cameras, optical detectors, optical microscopy systems, electrical systems, thermocouples and thermistors, pressure sensors, etc. The sensor can include a colorimetric detection system in some cases, which may be external to the device, or microfabricated into the device in certain cases. Various non-limiting examples of sensors and sensor techniques include colorimetric detection, pressure or temperature measurements, spectroscopy such as infrared, absorption, fluorescence, UV/visible, FTIR (“Fourier Transform Infrared Spectroscopy”), or Raman; piezoelectric measurements; immunoassays; electrical measurements, electrochemical measurements (e.g., ion-specific electrodes); magnetic measurements, optical measurements such as optical density measurements; circular dichroism; light scattering measurements such as quasielectric light scattering; polarimetry; refractometry; chemical indicators such as dyes; or turbidity measurements, including nephelometry.

In one set of embodiments, a sensor in the device may be used to determine a condition of blood present within the device. For example, the sensor may indicate the condition of analytes commonly found within the blood, for example, O₂, K⁺, hemoglobin, Na⁺, glucose, or the like. As a specific non-limiting example, in some embodiments, the sensor may determine the degree of hemolysis within blood contained within the device. Without wishing to be bound by any theory, it is believed that in some cases, hemolysis of red blood cells may cause the release of potassium ions and/or free hemoglobin into the blood. By determining the levels of potassium ions, and/or hemoglobin (e.g., by subjecting the device and/or the blood to separate cells from plasma, then determining hemoglobin in the plasma using a suitable colorimetric assay), the amount of blood lysis or “stress” experienced by the blood contained within the device may be determined. Accordingly, in one set of embodiments, the device may indicate the usability of blood (or other fluid) contained within the device, e.g., by indicating the degree of stress or the amount of blood lysis. Other examples of devices suitable for indicating the usability of blood (or other fluid) contained within the device are also discussed herein (e.g., by indicating the amount of time blood has been contained in the device, the temperature history of the device, etc.).

In some embodiments, an analyte may be determined as an “on/off” or “normal/abnormal” situation. Detection of the analyte, for example, may be indicative that insulin is needed; a trip to the doctor to check cholesterol; ovulation is occurring; kidney dialysis is needed; drug levels are present (e.g., especially in the case of illegal drugs) or too high/too low (e.g., important in care of geriatrics in particular in nursing homes). As another embodiment, however, an analyte may be determined quantitatively.

In one set of embodiments, the sensor may be a test strip, for example, test strips that can be obtained commercially. Examples of test strips include, but are not limited to, glucose test strips, urine test strips, pregnancy test strips, or the like. A test strip will typically include a band, piece, or strip of paper or other material and contain one or more regions able to determine an analyte, e.g., via binding of the analyte to a diagnostic agent or a reaction entity able to interact with and/or associate with the analyte. For example, the test strip may include various enzymes or antibodies, glucose oxidase and/or ferricyanide, or the like. The test strip may be able to determine, for example, glucose, cholesterol, creatinine, ketones, blood, protein, nitrite, pH, urobilinogen, bilirubin, leucocytes, luteinizing hormone, etc., depending on the type of test strip. The test strip may be used in any number of different ways. In some cases, a test strip may be obtained commercially and inserted into the device, e.g., before or after withdrawing blood or other fluids from a subject. At least a portion of the blood or other fluid may be exposed to the test strip to determine an analyte, e.g., in embodiments where the device uses the test strip as a sensor so that the device itself determines the analyte. In some cases, the device may be sold with a test strip pre-loaded, or a user may need to insert a test strip in a device (and optionally, withdraw and replace the test strip between uses). In certain cases, the test strip may form an integral part of the device that is not removable by a user. In some embodiments, after exposure to the blood or other fluid withdrawn from the subject, the test strip may be removed from the device and determined externally, e.g., using other apparatuses able to determine the test strip, for example, commercially-available test strip readers.

Other components may be present within the device, in some embodiments. For example, the device may contain a cover, displays, ports, transmitters, sensors, microfluidic channels, chambers, fluid channels, and/or various electronics, e.g., to control or monitor fluid transport into or out of the device, to determine an analyte present within a fluid delivered and/or withdrawn from the skin, to determine the status of the device, to report or transmit information regarding the device and/or analytes, or the like.

In some aspects, the device may include channels such as microfluidic channels, which may be used to move fluids within the device. In some cases, the microfluidic channels are in fluid communication with a needle that is used to deliver and/or withdraw fluids to or from the skin. For example, in one set of embodiments, the device may also include one or more microfluidic channels to contain fluid for delivery to the needle, e.g., from a source of fluid, and/or to withdraw fluid from the skin, e.g., for delivery to an analytical chamber within the device, to a reservoir for later analysis, or the like.

In some cases, more than one chamber may be present within the device, and in some cases, some or all of the chambers may be in fluidic communication, e.g., via channels such as microfluidic channels. In various embodiments, a variety of chambers and/or channels may be present within the device, depending on the application. For example, the device may contain chambers for sensing an analyte, chambers for holding reagents, chambers for controlling temperature, chambers for controlling pH or other conditions, chambers for creating or buffering pressure or vacuum, chambers for controlling or dampening fluid flow, mixing chambers, storage chambers for containing a fluid (e.g., withdrawn using a needle), drug chambers, or the like.

For instance, in some cases, the device may contain one or more chambers for holding or containing a fluid. In some cases, the chambers may be in fluidic communication with one or more fluid transporters and/or one or more microfluidic channels. For instance, the device may contain a chamber for containing fluid withdrawn from a subject (e.g., for storage and/or later analysis), a chamber for containing a fluid for delivery to the subject (e.g., blood, saline, optionally containing drugs, hormones, vitamins, pharmaceutical agents, or the like), etc.

In some cases, a storage chamber may contain a reagent or a reaction entity able to react with an analyte suspected of being present in the blood (or other fluid) entering the device, and in some cases, the reaction entity may be determined to determine the analyte. In some cases, the determination may be made externally of the device, e.g., by determining a color change or a change in fluorescence, etc. The determination may be made by a person, or by an external apparatus able to analyze at least a portion of the device. In some cases, the determination may be made without removing blood from the device, e.g., from the storage chamber. (In other cases, however, blood or other fluid may first be removed from the device before being analyzed.) For example, the device may include one or more sensors (e.g., ion sensors such as K+ sensors, colorimetric sensors, fluorescence sensors, etc.), and/or contain “windows” that allow light to penetrate the device. The windows may be formed of glass, plastic, etc., and may be selected to be at least partially transparent to one or a range of suitable wavelengths, depending on the analyte or condition to be determined. As a specific example, the entire device (or a portion thereof) may be mounted in an external apparatus, and light from the external apparatus may pass through or otherwise interact with at least a portion of the device (e.g., be reflected or refracted via the device) to determine the analyte and/or the reaction entity.

In some cases, the device may be designed such that portions of the device are separable. For example, a first portion of the device may be removed from the surface of the skin, leaving other portions of the device behind on the skin. In one embodiment, a stop may also be included to prevent or control the depth to which the needles or microneedles (or other fluid transporter components) are inserted into the skin, e.g., to control penetration to the epidermis, dermis, etc. As another example, a device may be modular, or include a portion that is removable from the device. For instance, blood or other bodily fluid may be received by the device in a portion (e.g., containing a storage chamber) that can be removed from the device. For instance, the removed portion can be stored, shipped to another location for analysis, or the like.

In one set of embodiments, the device contains a vacuum chamber that is also used as a storage chamber to receive blood or other fluid withdrawn from the skin of the subject into the device. For instance, blood withdrawn from a subject through or via the fluid transporter may enter the vacuum chamber due to its negative pressure (i.e., because the chamber has an internal pressure less than atmospheric pressure), and optionally stored in the vacuum chamber for later use. The fluid collected by the device can then be analyzed within the device or removed from the device for analysis, storage, etc.

In another set of embodiments, however, the device may include separate vacuum chambers and storage chambers (e.g., chambers to store fluid such as blood from the skin of the subject). The vacuum chamber and storage chambers may be in fluid communication, and may have any suitable arrangement. In some embodiments, the vacuum from the vacuum chamber may be used, at least in part, to withdraw fluid from the skin, which is then directed into a storage chamber, e.g., for later analysis or use, for example, as discussed below. As an example, blood may be withdrawn into the device, flowing towards a vacuum chamber, but the fluid may be prevented from entering the vacuum chamber. For instance, in certain embodiments, a material permeable to gas but not to a liquid such as blood may be used. For example, the material may be a membrane such as a hydrophilic or hydrophobic membrane having a suitable porosity, a porous structure, a porous ceramic frit, a dissolvable interface (e.g., formed from a salt or a polymer, etc.), or the like.

In some cases, the devices described herein can be single-stage or multi-stage. That is, the device can define a single unit that includes one or more components integrally connected to each other which cannot readily be removed from each other by a user, or can include one or more components which are designed to be and can readily be removed from each other. As a non-limiting example of the later, a two-stage device can be provided for application to the skin of a subject. The device can include a first portion designed to reside proximate the skin of the subject for the duration of the analysis, which might include an analysis region, a reservoir or other material for creating vacuum or otherwise promoting the flow of fluid or other materials relative to the analysis region, a needle or a microneedle to access interstitial fluid or blood, or the like. A second stage or portion of the device can be provided that can initiate operation of the device.

For example, the two-stage device can be applied to the skin of the user. A button or other component or switch associated with the second portion of the device can be activated by the subject to cause insertion of a needle or a microneedle to the skin of the subject, or the like. Then, the second stage can be removed, e.g., by the subject, and the first stage can remain on the skin to facilitate analysis.

In another example, a two-stage device can be provided where the first stage or portion includes visualization or other signal-producing components and the second stage or portion includes components necessary to facilitate the analysis, e.g., the second stage or portion can include all components necessary to access bodily fluid, transport the fluid (if necessary) to a site of analysis, and the like, and that stage can be removed, leaving only a visualization stage for the subject or another entity to view or otherwise analyze as described herein.

In yet another example, a two-stage device can include a first stage or portion that is applied to the skin of the subject, and a second stage or portion that stores blood or another bodily fluid. The second stage can be removed and stored, shipped to another location for analysis, or the like.

In certain embodiments, portions of the device may be constructed and arranged to be connectable and/or detachable from each other readily, e.g., by the subject. Thus, for instance, the subject (or another person) may be able to connect the portions (e.g., modules) to assemble a device, and/or disconnect the portions, without the use of tools such as screwdrivers or tape. In some cases, the connection and/or disconnection can occur while the device is affixed to the skin. Thus, for example, a device may be applied to the subject of the skin, and after use, a portion of the device may be removed from the skin of the subject, leaving the remainder of the device in place on the skin. Optionally, the portion may be replaced by another portion of the device, which may be the same or different than the removed portion.

As an example, in one embodiment, a device may be fabricated to contain a first module, and a second module that is constructed and arranged for repeated connection and disconnection to the first module. The first module may, for example, be used to deliver to and/or withdraw fluid from a subject. For instance, as discussed herein, the first module may contain a fluid transporter for delivering to and/or withdrawing fluid from the skin and/or beneath the skin of the subject. The fluid may optionally be analyzed within the first module, and/or stored for later use, e.g., in a collection chamber. After withdrawal of sufficient fluid, the first module may be removed, leaving the second module in place, and optionally replaced with a new first module for subsequent use (e.g., for subsequent delivery and/or withdrawal of fluid at a later time). In other embodiments, however, the second module may be removed, leaving the first module in place. Depending on the application, the removed module may be reused or disposed of (e.g., thrown in the trash), or the module may be shipped to another location for disposal and/or analysis, for example, to analyze fluid contained within the module, e.g., withdrawn from the skin of the subject. A module may be used once, or multiple times, before being removed from the device, depending on the application. Thus, as non-limiting examples the device may contain removable modules containing removable fluid transporters (e.g., needles or microneedles), removable modules for containing blood or another fluid, e.g., which can be shipped to another location, or the like.

In some aspects, any of the following components may independently be modular and may be single-use or reusable, or even absent in some cases: a pressure regulator such as a vacuum chamber or other vacuum source, an actuator, an activator, a fluid transporter, a fluid assay, a sensor, a fluid storage (e.g., a collection chamber), a data storage or memory component, a processor, a detector, a power source, a transmitter, a display, or the like. As non-limiting examples, one module could be a single use module (e.g., modules containing one or more of the following: fluid transporter, actuator, vacuum source, fluid processing, fluid storage, assay chemistry, etc.), or a module could be a re-usable module (e.g., modules containing one or more of the following: detector, processor, data storage, display, transmitter, power source, etc.) could be a re-useable module. Alternatively, just a single unit (e.g., a fluid transporter, e.g., one or more needles or microneedles) might be single use, and the rest of the device might be re-useable. Other combinations of these are also contemplated. In certain embodiments, the replaceable portion within the device is one that is required for the device to function, for example the device may not be able to function to deliver and/or withdraw fluid without the replaceable portion being present within the device. In one embodiment, the replaceable portion is not a power source (e.g., a battery).

In one set of embodiments, the device, or a portion thereof (e.g., a module) is reusable. For instance, the device may be used repeatedly (at the same location on the skin of a subject, or at different locations) to deliver to and/or withdraw fluid from the skin and/or beneath the skin of the subject. The device used repeatedly may be a single, integral device, and/or the device may contain one or more modules such as those previously discussed. For example, in some cases, between uses, a module may be removed and/or replaced from the device, e.g., as discussed above.

In one set of embodiments, a device as discussed herein (or a portion thereof) may be shipped or transported to another location for analysis. For example, the device or a module may be hand-carried, mailed, etc. In some cases, the device may include an anticoagulant or a stabilizing agent contained within the device, e.g., within a storage chamber for the fluid. Thus, for example, fluid such as blood withdrawn from the skin may be delivered to a chamber (e.g., a storage chamber) within the device, then the device, or a portion of the device (e.g., a module) may be shipped to another location for analysis. Any form of shipping or transport may be used, e.g., via mail or hand-delivery.

After withdrawal of the fluid into the device, the device, or a portion thereof, may be removed from the skin of the subject, e.g., by the subject or by another person. For example, the entire device may be removed, or a portion of the device containing the storage reservoir may be removed from the device, and optionally replaced with another storage reservoir. Thus, for instance, in one embodiment, the device may contain two or more modules, for example, a first module that is able to cause withdrawal of fluid from the skin into a storage reservoir, and a second module containing the storage module. In some cases, the module containing the storage reservoir may be removed from the device.

As another example, the device may include at least two modules manually separable from each other, including a first module comprising a vacuum chamber, and a second module comprising other components such as those described herein. In some embodiments, the modules may be separable without the use of tools. For example, the second module may include one or more components such as a fluid transporter (e.g., a needle or microneedle), an applicator region such as a recess, a reversibly deformable structure such as a flexible concave member, a collection chamber, a sensor, a processor, or the like. As a specific example, the first module may be defined entirely or partially by a vacuum chamber, and the first module may be removed and replaced with a fresh vacuum chamber, during or between uses. Thus, for instance, the first module may be inserted into the device when blood or other bodily fluids are desired to be withdrawn from a subject, and optionally, used to cause blood to be withdrawn from the skin of the subject.

In one set of embodiments, the first module may be substantially cylindrical, and in some embodiments, the first module may be a Vacutainer™ tube, a Vacuette™ tube, or other commercially-available vacuum tube, or other vacuum source such as is described herein. In some embodiments, a Vacutainer™ or Vacuette™ tube that is used may have a maximum length of no more than about 75 mm or about 100 mm and a diameter of no more than about 16 mm or about 13 mm. The device, in certain embodiments, may also contain an adaptor able to hold or immobilize such tubes on the device, for example, a clamp. Other examples of adaptors are discussed in detail herein. In some cases, the device may have a shape or geometry that mimics a Vacutainer™ or Vacuette™ tube, e.g., one having the above dimensions. The device, in some embodiments, is substantially cylindrically symmetric.

The withdrawn fluid may then be sent to a clinical and/or laboratory setting, e.g., for analysis. In some embodiments, the entire device may be sent to the clinical and/or laboratory setting; in other embodiments, however, only a portion of the device (e.g., a module containing a storage reservoir containing the fluid) may be sent to the clinical and/or laboratory setting. In some cases, the fluid may be shipped using any suitable technique (e.g., by mail, by hand, etc.). In certain instances, the subject may give the fluid to appropriate personnel at a clinical visit. For instance, a doctor may prescribe a device as discussed above for use by the subject, and at the next doctor visit, the subject may give the doctor the withdrawn fluid, e.g., contained within a device or module.

One aspect is directed to an adaptor able to position a device in apparatuses designed to contain Vacutainer™ tubes or Vacuette™ tubes. In some cases, the Vacutainer™ or Vacuette™ tube sizes have a maximum length of no more than about 75 mm or about 100 mm and a diameter of no more than about 16 mm or about 13 mm. In some cases, the adaptor may be able to immobilize a device therein, e.g., for subsequent use or processing. In some cases, devices may have a largest lateral dimension of no more than about 50 mm, and/or a largest vertical dimension, extending from the skin of the subject when the device is applied to the subject, of no more than about 10 mm. The device may contained within the adaptor using any suitable technique, e.g., using clips, springs, braces, bands, or the application of force to the device present within the adaptor.

According to one aspect, the device is of a relatively small size. For example, in some embodiments, the device may have a largest lateral dimension (e.g., parallel to the skin) of no more than about 25 cm, no more than about 10 cm, no more than about 7 cm, no more than about 6 cm, no more than about 5.5 cm, no more than about 5 cm, no more than about 4.5 cm, no more than about 4 cm, no more than about 3.5 cm, no more than about 3 cm, no more than about 2 cm, or no more than about 1 cm. In some cases, the device may have a largest lateral dimension of between about 0.5 cm and about 1 cm, between about 2 and about 3 cm, between about 2.5 cm and about 5 cm, between about 2 cm and about 7 cm, etc.

In some embodiments, the device is relatively lightweight. For example, the device may have a mass of no more than about 1 kg, no more than about 300 g, no more than about 150 g, no more than about 100 g, no more than about 50 g, no more than about 30 g, no more than about 25 g, no more than about 20 g, no more than about 10 g, no more than about 5 g, or no more than about 2 g. For instance, in various embodiments, the device has a mass of between about 2 g and about 25 g, a mass of between about 2 g and about 10 g, a mass of between 10 g and about 50 g, a mass of between about 30 g and about 150 g, etc.

Combinations of these and/or other dimensions are also possible in other embodiments. As non-limiting examples, the device may have a largest lateral dimension of no more than about 5 cm, a largest vertical dimension of no more than about 1 cm, and a mass of no more than about 25 g; or the device may have a largest lateral dimension of no more than about 5 cm, a largest vertical dimension of no more than about 1 cm, and a mass of no more than about 25 g; etc. As additional non-limiting examples, the device may have dimensions of no more than 2.0 cm×3.1 cm×5.7 cm (height×width×length), no more than 2.5 cm×3.5 cm×6.0 cm, no more than about 1.5 cm×4.2 cm×4.7 cm, no more than 2.0 cm×4.5 cm×5.0 cm, etc.

In some embodiments, the device may be sized such that it is wearable and/or able to be carried by a subject. For example, the device may be self-contained, needing no wires, cables, tubes, external structural elements, or other external support. The device may be positioned on any suitable position of the subject, for example, on the arm or leg, on the back, on the abdomen, etc.

In some embodiments, the device may be connected to an external apparatus for determining at least a portion of the device, a fluid removed from the device, an analyte suspected of being present within the fluid, or the like. For example, the device may be connected to an external analytical apparatus, and fluid removed from the device for later analysis, or the fluid may be analyzed within the device in situ, e.g., by adding one or more reaction entities to the device, for instance, to a storage chamber, or to analytical chamber within the device. For example, in one embodiment, the external apparatus may have a port or other suitable surface for mating with a port or other suitable surface on the device, and blood or other fluid can be removed from the device using any suitable technique, e.g., using vacuum or pressure, etc. The blood may be removed by the external apparatus, and optionally, stored and/or analyzed in some fashion. For example, in one set of embodiments, the device may include an exit port for removing a fluid from the device (e.g., blood). In some embodiments, fluid contained within a storage chamber in the device may be removed from the device, and stored for later use or analyzed outside of the device. In some cases, the exit port may be separate from the fluid transporter.

In one aspect, the device may be interfaced with an external apparatus able to determine an analyte contained within a fluid in the device, for example within a storage chamber as discussed herein. For example, the device may be mounted on an external holder, the device may include a port for transporting fluid out of the device, the device may include a window for interrogating a fluid contained within the device, or the like.

In some embodiments, the device may be connected to an external apparatus for determining at least a portion of the device, a fluid removed from the device, an analyte suspected of being present within the fluid, or the like. For example, the device may be connected to an external analytical apparatus, and fluid removed from the device for later analysis, or the fluid may be analyzed within the device in situ, e.g., by adding one or more reaction entities to the device, for instance, to a storage chamber, or to analytical chamber within the device. For example, in one embodiment, the external apparatus may have a port or other suitable surface for mating with a port or other suitable surface on the device, and blood or other fluid can be removed from the device using any suitable technique, e.g., using vacuum or pressure, etc. The blood may be removed by the external apparatus, and optionally, stored and/or analyzed in some fashion. For example, in one set of embodiments, the device may include an exit port for removing a fluid from the device (e.g., blood). In some embodiments, fluid contained within a storage chamber in the device may be removed from the device, and stored for later use or analyzed outside of the device. In some cases, the exit port may be separate from the fluid transporter. For example, an exit port can be in fluidic communication with a vacuum chamber, which can also serve as a fluid reservoir in some cases. Other methods for removing blood or other fluids from the device include, but are not limited to, removal using a vacuum line, a pipette, extraction through a septum instead of an exit port, or the like. In some cases, the device may also be positioned in a centrifuge and subjected to various g forces (e.g., to a centripetal force of at least 50 g), e.g., to cause at separation of cells or other substances within a fluid within the device to occur.

In some cases, the device may include a drug or a therapeutic agent for delivery to a subject. For example, the drug may include an anti-inflammatory compound, an analgesic, or an anti-histamine compound. Examples of anti-inflammatory compounds include, but are not limited to, NSAIDs (non-steroidal anti-inflammatory drugs) such as aspirin, ibuprofen, or naproxen. Examples of analgesics include, but are not limited to, benzocaine, butamben, dibucaine, lidocaine, oxybuprocaine, pramoxine, proparacaine, proxymetacaine, tetracaine, acetaminophen, NSAIDs such as acetylsalicylic acid, salicylic acid, diclofenac, ibuprofen, etc., or opioid drugs such as morphine or opium, etc. Examples of anti-histamine compounds include, but are not limited to, clemastine, diphenhydramine, doxylamine, loratadine, desloratadine, fexofenadine, pheniramine, cetirizine, ebastine, promethazine, chlorpheniramine, levocetirizine, olopatadine, quetiapine, meclizine, dimenhydrinate, embramine, dimethindene, dexchlorpheniramine, vitamin C, cimetidine, famotidine, ranitidine, nizatidine, roxatidine, or lafutidine. Other specific non-limiting examples of therapeutic agents that could be used include, but are not limited to biological agents such as erythropoietin (“EPO”), alpha-interferon, beta-interferon, gamma-interferon, insulin, morphine or other pain medications, antibodies such as monoclonal antibodies, or the like.

As mentioned, the device may include an anticoagulant or a stabilizing agent for stabilizing the fluid withdrawn from the skin. As a specific non-limiting example, an anticoagulant may be used for blood withdrawn from the skin. For example, the anticoagulant or stabilizing agent may be present within a storage chamber of the device.

Examples of anticoagulants include, but are not limited to, heparin, citrate, oxalate, or ethylenediaminetetraacetic acid (EDTA). Other agents may be used in conjunction or instead of anticoagulants, for example, stabilizing agents such as solvents, diluents, buffers, chelating agents, antioxidants, binding agents, preservatives, antimicrobials, or the like. Examples of preservatives include, for example, benzalkonium chloride, chlorobutanol, parabens, or thimerosal. Non-limiting examples of antioxidants include ascorbic acid, glutathione, lipoic acid, uric acid, carotenes, alpha-tocopherol, ubiquinol, or enzymes such as catalase, superoxide dismutase, or peroxidases. Examples of microbials include, but are not limited to, ethanol or isopropyl alcohol, azides, or the like. Examples of chelating agents include, but are not limited to, ethylene glycol tetraacetic acid or ethylenediaminetetraacetic acid. Examples of buffers include phosphate buffers such as those known to ordinary skill in the art.

The device may be used with an analgesic or other agent that alters or inhibits sensation. For example, an analgesic such as benzocaine, butamben, dibucaine, lidocaine, oxybuprocaine, pramoxine, proparacaine, proxymetacaine, or tetracaine may be applied to the skin, prior to or during delivery and/or withdrawal of fluid, or another obscuring agent may be applied, e.g., an agent to cause a burning sensation, such as capsaicin or capsaicin-like molecules, for example, dihydrocapsaicin, nordihydrocapsaicin, homodihydrocapsaicin, homocapsaicin, or nonivamide. Further examples of analgesics include, but are not limited to, acetaminophen, NSAIDs such as acetylsalicylic acid, salicylic acid, diclofenac, ibuprofen, etc., or opioid drugs such as morphine or opium, etc.

The analgesic or other agent may be applied to the skin using any suitable technique, e.g., using the device, or separately. The analgesic or other agent may be applied to the skin automatically, or upon activation of the device as discussed herein. For example, the analgesic or other agent may be delivered to the skin (e.g., via a microfluidic channel from a chamber containing the analgesic or other agent) prior to, and/or after, exposure of the skin to a fluid transporter as discussed herein. In some cases, the analgesic or other agent may be sprayed on the skin, e.g., through a nozzle. In another embodiment, a sponge, gauze, a swab, a membrane, a filter, a pad, or other absorbent material may be applied to the skin (e.g., by the device) to apply the analgesic or other agent to the skin, e.g., to blood or other bodily fluids present on the skin. In some cases, a fluid transporter may pass through the material. For example, upon application of the device to the skin, a portion of the device (e.g., a cover) may be moved, thereby exposing the skin to material contained within the device that contains the analgesic or other agent to be applied to the skin. In some cases, an applicator, such as a brush, a pad, or a sponge, may be moved on the surface of the skin to apply the analgesic or other agent the skin. For example, the device may move an applicator across the surface of the skin.

In one aspect, the device may include a system for sanitizing at least a portion of the skin of the subject, for example, the region of skin where fluid is delivered and/or withdrawn. The region may be sanitized at any suitable time. For instance, the region may be sanitized before, during, and/or after delivery to and/or withdrawal of fluid from the skin and/or beneath the skin of the subject. In some embodiments, the system sanitizing the skin may be formed as an integral part of the device; in other embodiments, however, the system may be contained within a module that is connectable and/or detachable to the remainder of the device (e.g., to other modules within the device). For example, the device may contain a sterilization module that optionally can be removed from the device and/or replaced with a new sterilization module, in various embodiments.

As used herein, “sanitizing” means that at least some of the microorganisms present on the surface of the skin are killed and/or inactivated (e.g., rendered uninfectious). The microorganisms that may be present include, for example, bacteria (e.g., of the genuses Propionibacteria, Corynebacteria, Staphylococcus, and/or Streptococcus, etc.), fungi, viruses (e.g., coronaviruses, such as SARS-CoV-2), or the like. It should be understood, however, that the skin may be “sanitized” without necessarily killing 100% of the microorganisms present on the skin in the region being sanitized. For example, the sanitization system may be effective at killing and/or inactivating at least 25%, at least 50% or at least 75% of the microorganisms, or by killing and/or inactivating the microorganisms by 1, 2, 3, or 4 logs, where a “log” is a 10-fold reduction in the number of active microorganisms.

In one set of embodiments, the device contains a fluid containing a sanitizer, and the fluid is applied to the skin. The fluid may be applied to the skin automatically, or upon activation of the device as discussed herein. For example, the fluid may be delivered to the skin (e.g., via a microfluidic channel from a chamber containing the fluid) prior to, and/or after, exposure of the skin to a fluid transporter as discussed herein. In some cases, the fluid may be sprayed on the skin, e.g., through a nozzle. In another embodiment, a sponge, gauze, a swab, a membrane, a filter, a pad, or other absorbent material may be applied to the skin (e.g., by the device) to sanitize the skin. In some cases, a fluid transporter may pass through the material. As another example, a portion of the device (e.g., a cover) may be moved, thereby exposing the skin to material contained within the device that contains the sanitizer. In some cases, an applicator, such as a pad, a brush or a sponge, may be moved on the surface of the skin to sanitize the skin. For example, the device may move an applicator across the surface of the skin.

In one set of embodiments, the sanitizer is a liquid, gel, or foam, and/or is contained in a liquid, gel, or foam. The sanitizer may be any suitable agent able to sanitize the skin, for example, a peroxide (e.g., H₂O₂), bleach, an alcohol (e.g., ethyl alcohol, isopropyl alcohol, etc.), n-propanol, triclosan, benzalkonium chloride, tincture of iodine (e.g., containing 2-7% potassium iodide or sodium iodide, and elemental iodine, dissolved in a mixture of ethanol and water), povidone-iodine (e.g., Betadine) chlorhexidine gluconate, or soap (e.g., common soap, such as liquid soap), or the like. In another set of embodiments, however, the sanitizer may take the form of a source of radiation, for example, ultraviolet radiation.

Other aspects are directed to a kit including one or more devices such as previously discussed. The kit may include a package or an assembly including one or more of the devices such as described herein, and/or other components associated with such devices, for example, as previously described. For example, in one set of embodiments, the kit may include a device and one or more compositions for use with the device. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit may, in some cases, include instructions in any form that are provided in connection with a device in such a manner that one of ordinary skill in the art would recognize that the instructions are to be associated with the device. For instance, the instructions may include instructions for using, modifying, storing, shipping, repairing, dissembling, etc. the device. In some cases, the instructions may also include the use, modification, mixing, diluting, preserving, administering, assembly, storage, packaging, and/or preparation of the compositions and/or other compositions associated with the kit. In some cases, the instructions may also include instructions for the delivery and/or administration of the device, for example, for a particular use, e.g., to a subject. The instructions may be provided in any form recognizable as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, a fluid receiving device may include features that are generally directed to separating blood into plasma or serum, and a portion enriched in blood cells, for example, under vacuum or reduced pressure. For example, a device may draw blood (or other suitable bodily fluids) into the device and/or through a membrane, such as a separation membrane. In some embodiments, the membrane is used to separate the blood into a first portion formed of plasma or serum, and a second portion that is concentrated in blood cells.

In some cases, the device may be used to separate a relatively small amount of blood into plasma or serum and a portion concentrated in blood cells. For example, less than about 10 ml, less than about 5 ml, less than about 3 ml, less than about 2 ml, less than about 1.5 ml, less than about 1 ml, less than about 800 microliters, less than about 600 microliters, less than about 500 microliters, less than about 400 microliters, less than about 300 microliters, less than about 200 microliters, less than about 100 microliters, less than about 80 microliters, less than about 60 microliters, less than about 40 microliters, less than about 20 microliters, less than about 10 microliters, or less than about 1 microliter of blood may be received into the device and separated within the device. The plasma or serum can then be recovered from the device, for example, using a needle to remove at least a portion of the plasma or serum, and subjected to various diagnostics or testing protocols, for example, for the detection of infections, diabetes (e.g., sugar), AIDS (e.g., HIV), cancer (e.g., prostate-specific antigen), or other indications. In some embodiments, the device may be relatively small, in contrast with machines (such as dialysis machines) that are typically used in plasmapheresis. For example, the device may be handheld or be applied to the skin of a subject, e.g., using an adhesive, as is discussed below. The device may be self-contained in some embodiments, i.e., such that the device is able to function to withdraw blood (or other bodily fluids) from a subject and separate it to produce plasma or serum without requiring external connections such as an external source of vacuum, an external source of power, or the like. For instance, a vacuum source within the device, e.g., a vacuum chamber, may be used to draw blood across the separation membrane to produce plasma or serum.

Furthermore, in certain embodiments, the device is able to effectively produce a relatively small amount of plasma or serum without requiring a relatively large amount of blood and/or without requiring a centrifuge to produce plasma or serum from the received blood. In some cases, at least about 30%, at least about 40%, at least about 50%, at least about 60%, at least about 70%, at least about 80%, or at least about 90% of the plasma or serum produced by the device may be received from the device, e.g., for use in subsequent testing or diagnostics. In contrast, in many prior art techniques where a sample of plasma or serum is required, e.g., for diagnostics or testing purposes, a relatively large volume of blood is received from a subject into a test tube (e.g., having a volume of at least 2 ml, at least 4 ml, at least 6 ml, or at least about 10 ml, such as in the Vacutainer™ (Becton, Dickinson and company) or Vacuette™ (Greiner Bio-One GmBH) systems), then the test tube is processed (for example, via centrifugation) to separate the blood from the plasma or serum. A portion of the plasma or serum is then removed from the test tube for diagnostics or testing purposes; however, the remainder of the plasma or serum within the test tube is not needed for subsequent testing or diagnostics, and is essentially wasted. Additionally, in some embodiments, serum may be produced without use of an anticoagulant within the device, although in other embodiments, the device may contain an anticoagulant to produce plasma. In some embodiments, the membrane and/or the storage chamber may contain an anticoagulant to produce plasma. Alternatively, if there is no anticoagulant present in the device, fluid that flows through a separation membrane into the storage chamber is free of blood cells and will ultimately clot in the storage chamber, thereby producing a liquid component, also known as serum. This serum can be collected via aspiration or other suitable method out of the storage chamber, leaving the blood clots in the storage chamber. Thus, many embodiments described herein may be used to produce plasma or serum, depending on the presence or absence of anticoagulant.

As mentioned, in one aspect, blood received from a subject into a device may be separated within the device to form plasma or serum by passing the blood, or at least a portion thereof, through a separation membrane or a membrane that is permeable to fluids but is substantially impermeable to cells. The separation membrane can be any membrane able to separate blood passing therethrough into a first portion (passing through the membrane) that is enriched in plasma or serum, and a second portion (rejected by the membrane) concentrated in blood cells. In some cases, the separation membrane may have a separation effectiveness during use (the separation effectiveness is the volume of plasma or serum that passes through the membrane relative to the starting volume of whole blood) of at least about 5%, at least about 10%, at least about 20%, at least about 40%, at least about 50%, at least about 55%, or at least about 60%.

In one set of embodiments, the separation membrane is selected to have a pore size smaller than the average or effective diameter of blood cells contained within the blood, including red blood cells and white blood cells. For instance, the pore size of the separation membrane may be less than about 30 micrometers, less than about 20 micrometers, less than about 10 micrometers, less than about 8 micrometers, less than about 6 micrometers, less than about 4 micrometers, less than about 3 micrometers, less than about 2 micrometers, less than about 1.5 micrometers, less than about 1 micrometer, less than about 0.5 micrometers, etc. As specific non-limiting examples, the pore size may be between about 0.5 micrometers and about 2 micrometers, or between about 0.5 micrometers and about 1 micrometer. In addition, in some embodiments, the separation membrane may have a thickness of less than about 1 mm, less than about 750 micrometers, less than about 500 micrometers, less than about 400 micrometers, less than about 350 micrometers, less than about 300 micrometers, less than about 250 micrometers, or less than about 200 micrometers.

The separation membrane may be formed out of any suitable material. For example, in some embodiments, the separation membrane may be formed out of a material that promotes thrombolysis or inhibits clot formation, such as a polyester, and/or the separation membrane may be formed and/or coated with a biocompatible material, or at least a material that does not cause an active clotting response within the blood that the separation membrane is exposed to. As specific non-limiting example, the separation membrane can comprise or be formed from glass (e.g., glass fibers), and/or a polymer such as a polycarbonate, a polysulfone, a polyethersulfone, a polyarylethersulfone, a polyvinylpyrrolidone, a polypropylene, poly(2-methoxyethylacrylate), and/or a nitrocellulose, etc. In some embodiments, the membrane may include a copolymer such as a graft copolymer (for example, poly(propylene-graft-2-methoxyethylacrylate)), e.g., including any one or more of these polymers and/or other suitable polymers. In some cases, the separation membrane may be asymmetric, e.g., having a different separation effectiveness depending on which way blood is passed across the separation membrane to produce plasma. Many such separation membranes may be readily obtained commercially, such as Pall Vivid Plasma Separation Membrane (GF, GX, and GR), as well as other commercially available separation membranes.

During use, blood is moved towards the separation membrane using a suitable driving force to move the blood, for example, vacuum or other reduced pressure as is discussed herein. A fluidic portion of the blood is able to pass across the separation membrane to form plasma or serum on one side of the membrane, while other portions of the blood, e.g., red and white blood cells, are rejected by the membrane and thus form a portion that becomes concentrated in blood cells. For example, serum may be produced if no anticoagulant is present, in accordance with certain embodiments. Either or both portions of the blood may be collected, e.g., in an appropriate storage chamber, for further use, analysis, storage, etc., as is discussed herein.

In some embodiments, a fluid receiving device may include features that are generally directed to substrates for absorbing blood and/or other bodily fluids, for example, a blood spot membrane. Thus, in some embodiments, blood spots may be produced on a blood spot membrane. In these cases, a channel within the device may have a small volume relative to the volume of a blood spot membrane which may be very porous and may collect fluid. The blood spot membrane is used to collect fluid in certain embodiments. The blood spot membrane is not used to separate cells/plasma (as opposed to the separation membranes discussed herein), in certain cases. Fluid may fill all, or a portion of, the blood spot membrane. A second hydrophobic membrane may be positioned on top of the collection membrane in some embodiments. Once fluid contacts the hydrophobic membrane, fluid collection may cease. The blood spot membrane may remain in the device to dry and can then be removed from the device. In some embodiments, the blood spot membrane may be removed from the device and dried outside of the device. In some cases, the membrane is not dried. If a vacuum is used to draw blood towards the blood spot membrane, the vacuum may be released prior to removal of the blood spot membrane from the device, at least in some embodiments.

In one set of embodiments, the substrate is contained within a device for receiving blood from the skin of a subject. Examples of such devices, and details of such devices able to contain a substrate for absorbing blood and/or other bodily fluids, are discussed in detail below.

In one set of embodiments, the substrate for absorbing blood may comprise paper, e.g., that is able to absorb blood or other bodily fluids received by the device. The substrate may be able to partially or wholly absorb any blood (or other bodily fluid) that it comes into contact with. For example, the substrate may comprise filter paper, cellulose filters, cotton-based paper, e.g., made from cellulose filters, cotton fibers (e.g., cotton linters), glass fibers, or the like. Specific non-limiting examples that are commercially available include Schleicher & Schuell 903™ or Whatman 903™ paper (Whatman 903™ Specimen Collection Paper) (Whatman International Limited, Kent, UK), or Ahlstrom 226 specimen collection paper (Ahistrom Filtration LLC, Mount Holly Springs, PA). In some embodiments, the paper may be one that is validated in compliance with the requirements of the CLSI (Clinical and Laboratory Standards Institute) LA4-A5 consensus standard. However, other materials may also be used for the substrate for absorbing blood, instead of and/or in addition to paper. For example, the substrate for absorbing blood (or other bodily fluids) may comprise gauze, cloth, cardboard, foam, foamboard, paperboard, a polymer, a gel, or the like. In some cases, the absorbent substrate may have a surface area of at least about 0.001 m²/g, at least about 0.003 m²/g, at least about 0.005 m²/g, at least about 0.01 m²/g, at least about 0.03 m²/g, at least about 0.05 m²/g, at least about 0.1 m²/g, at least about 0.3 m²/g, at least about 0.5 m²/g, or at least about 1 m²/g. In some cases, the absorbent substrate may have a surface area of about 100 g/m² to about 200 g/m², or about 150 g/m² to about 200 g/m².

The blood (or other bodily fluid) may be absorbed into the substrate such that the blood becomes embedded within fibers or other materials forming the substrate, and/or such that the blood becomes embedded in spaces between the fibers or other materials forming the substrate. For example, the blood may be held within or on the substrate mechanically and/or chemically (e.g., via clotting and/or reaction with fibers or other materials forming the substrate).

In some cases, the substrate may absorb a relatively small amount of blood. For example, less than about 1 ml, less than about 800 microliters, less than about 600 microliters, less than about 500 microliters, less than about 400 microliters, less than about 300 microliters, less than about 200 microliters, less than about 100 microliters, less than about 80 microliters, less than about 60 microliters, less than about 40 microliters, less than about 30 microliters, less than about 20 microliters, less than about 10 microliters, or less than about 1 microliter of blood may be absorbed into the substrate.

The substrate may be of any shape or size. In some embodiments, the substrate may be substantially circular, although in other embodiments, other shapes are possible, e.g., square or rectangular. The substrate may have any suitable area. For example, the substrate may be large enough to contain only one spot, of blood (e.g., of the above volumes), or more than one spot in some embodiments. For example, the substrate may have an area of no more than about 1 cm², no more than about 3 cm², no more than about 5 cm², no more than about 7 cm², no more than about 10 cm², no more than about 30 cm², no more than about 50 cm², no more than about 100 cm², no more than about 300 cm², no more than about 500 cm², no more than about 1000 cm², or no more than about 3000 cm².

In some embodiments, a “tab” or a handle, or other separate portion, may be present on or proximate the substrate, e.g., to facilitate analysis and/or manipulation of the absorbed blood or other bodily fluid. The handle may be any portion that can be used to manipulate the substrate. For example, a handle may be used to remove the substrate from the device for subsequent shipping and/or analysis, e.g., without requiring a person to touch the blood spot itself in order to manipulate the substrate. The handle may be formed from the substrate, and/or different material, for example, plastic, cardboard, wood, metal, etc. In some cases, the handle may surround all, or at least a portion of, the substrate.

In certain embodiments, the substrate may include stabilizers or other agents, e.g., for stabilizing and/or treating the blood in the substrate. Non-limiting examples of stabilizers include chelating agents, enzyme inhibitors, or lysing agents. Examples of chelating agents include, but are not limited to, EDTA (ethylenediaminetetraacetic acid) or dimercaprol. Examples of enzyme inhibitors include, but are not limited to, protease inhibitors (e.g., aprotinin, bestatin, calpain inhibitor I and II, chymostatin, E-64, leupeptin or N-acetyl-L-leucyl-L-leucyl-L-argininal, alpha-2-macroglobuline, Pefabloc SC, pepstatin, PMSF or phenylmethanesulfonyl fluoride, TLCK, a trypsin inhibitor, etc.) or reverse transcriptase inhibitors (e.g., zidovudine, didanosine, zalcitabine, stavudine, lamivudine, abacavir, emtricitabine, entecavir, apricitabine, etc.). Non-limiting examples of lysing agents include distilled water or guanidinium thiocyanate.

A PCT application entitled “Devices and Methods for Receiving Fluids,” having a filing date of May 1, 2020, is incorporated herein by reference in its entirety. In addition, the following are each also incorporated herein by reference in their entireties: U.S. Pat. Apl. Ser. Nos. 62/842,303; 62/880,137; 62/942,540; 62/948,788; and 62/959,868.

While aspects of the disclosure have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the disclosure.

What is claimed is:

1. A vacuum generation device, comprising:
a chamber having an active volume that is decreasable from a first volume to a second volume due to application of force during a priming step having a first phase and a second phase, the chamber being biased to return toward the first volume when the application of force ceases, and a required application of force is higher in the first phase than in the second phase; and
a one-way vent, wherein air exits the chamber through the one-way vent as the active volume is decreased from the first volume to the second volume,
wherein return of the chamber toward the first volume from the second volume creates a vacuum.

2. The vacuum generation device of claim 1, wherein the chamber does not include a flexible dome that is biased to return toward a non-compressed state.

3. The vacuum generation device of claim 1 or 2, further comprising a spring that biases the chamber to return toward the first volume when the application of force ceases.

4. The vacuum generation device of claim 3, wherein the spring comprises a first spring and a second spring.

5. The vacuum generation device of claim 4, wherein the first spring and the second spring have different stiffnesses.

6. The vacuum generation device of claim 4, further comprising a locking arrangement that permits loading of the first spring and prohibits loading of the second spring during the first phase of the priming step, and wherein the locking arrangement permits loading of the second spring during the second phase of the priming step.

7. The vacuum generation device of claim 6, wherein the locking arrangement prohibits loading of the first spring during the second phase of the priming step.

8. The vacuum generation device of any one of the above claims, wherein the chamber comprises a syringe.

9. The vacuum generation device of claim 1 or 2, wherein the chamber comprises a body and a plunger moveable within the body, wherein a position of the plunger defines the volume of the active volume.

10. The vacuum generation device of claim 6, wherein the locking arrangement comprises a latch.

11. The vacuum generation device of claim 6, wherein the locking arrangement comprises a friction pad.

12. The vacuum generation device of claim 6, wherein the locking arrangement comprises a breakaway connection.

13. The vacuum generation device of claim 3, wherein the spring comprises at least one of a coil spring, a torsion spring, a pneumatic spring, stretchable elastomers, and a compressible foam.

14. The vacuum generation device of claim 1 or 2, further comprising a return element.

15. The vacuum generation device of claim 14, wherein the return element comprises at least one of a spring or a bi-stable dome.

16. The vacuum generation device of claim 6, wherein loading of the first spring comprises elongation or compression of the first spring, and loading of the second spring comprises elongation or compression of the second spring.

17. A method of generating vacuum, comprising:
applying a first force to decrease an active volume of a chamber from a first volume during a first phase of a priming step;
applying a second force to further decrease the active volume of the chamber during a second phase of the priming step, the first force being greater than the second force;
venting air out of the chamber during the priming step; and
ceasing application of force to permit the active volume of the chamber to return toward the first volume, wherein vacuum is generated due to the return of the active volume of the chamber toward the first volume.

18. The method of claim 17, wherein the chamber does not include a flexible dome that is biased to return toward a non-compressed state.

19. The method of claim 17, further comprising loading a first return element during the first phase of a priming step while prohibiting loading of a second return element, and loading the second return element during the second phase of the priming step while prohibiting loading and unloading of the first return element, wherein the first return element and the second return element are configured to bias the active volume back toward the first volume.

20. The method of claim 19, wherein the first return element comprises a first spring and the second return element comprises a second spring.

21. The method of claim 20, wherein the first spring has a higher stiffness than a stiffness of the second spring.

22. A vacuum generation device, comprising:
a chamber having an active volume that is decreasable from a first volume to a second volume due to application of force during a priming step having a first phase and a second phase;
a first return element that is loaded during the first phase of the priming step, the chamber being biased to return toward the first volume by the first return element when the application of force ceases;
a second return element that is loaded during the second phase of the priming step, the chamber being biased to return toward the first volume by the second return element when the application of force ceases, wherein a stiffness of the first return element is greater than a stiffness of the second return element; and
a one-way vent, wherein air exits the chamber through the one-way vent as the active volume is decreased from the first volume to the second volume,
wherein return of the chamber toward the first volume from the second volume creates a vacuum.

23. The vacuum generation device of claim 22, wherein the first return element comprises a spring.

24. The vacuum generation device of claim 23, wherein the spring comprises at least one of a coil spring, a torsion spring, a pneumatic spring, stretchable elastomers, and a compressible foam.

25. The vacuum generation device of claim 22, wherein the first return element comprises a bi-stable dome.

26. The vacuum generation device of claim 22, further comprising a locking arrangement that permits loading of the first return element and prohibits loading of the second return element during the first phase of the priming step, and wherein the locking arrangement permits loading of the second return element during the second phase of the priming step.

27. The vacuum generation device of claim 26, wherein the locking arrangement comprises at least one of a latch, a friction pad and a breakaway connection.

28. A method of generating vacuum, comprising:
loading a first return element while decreasing an active volume of a chamber from a starting volume during a first phase of a priming step;
loading a second return element while decreasing the active volume of a chamber during a second phase of a priming step, wherein a stiffness of the first return element is greater than a stiffness of the second return element;
venting air out of the chamber during the priming step; and
ceasing application of force to permit the active volume of the chamber to return toward the starting volume, wherein vacuum is generated in a vacuum generation step due to the return of the active volume of the chamber toward the starting volume.

29. The method of claim 28, wherein the chamber does not include a flexible dome that is biased to return toward a non-compressed state.

30. The method of claim 28, further comprising locking the second return element from being loaded while the first return element is loaded during the first phase of the priming step, and locking the first return element from being loaded and from being unloaded while the second return element is loaded during the second phase of the priming step.

31. The method of claim 30, further comprising locking the first return element from being unloaded while the second return element is unloaded during a first phase of the vacuum generation step, and locking the second return element from being loaded and from being unloaded while the first return element is unloaded during a second phase of the vacuum generation step.
